(19) United States
(12) Patent Application Publication
(10) Pub. No.: US 2009/0030012 A1
(43) Pub. Date: Jan. 29, 2009

(54) Pyridine, Pyrimidine and Pyrazine Derivatives as Cxcr3 Receptor Modulators

(76) Inventors: Alan D. Adams, Cranford, NJ (US); Conrad Santini, Stirling, NJ (US)

Correspondence Address: MERCK AND CO., INC, P O BOX 2000, RAHWAY, NJ, 07065-0907, US

(21) Appl. No.: 12/223,556

(22) PCT Filed: Feb. 20, 2007
(86) PCT No.: PCT/US07/04618

§ 371 Date: Aug. 1, 2008

Related U.S. Application Data
(60) Provisional application No. 60/775,874, filed on Feb. 23, 2006.

(51) Int. Cl. A61K 31/496; C07D 401/14; A61K 31/4545; A61P 19/02; A61P 37/06; A61P 17/06; A61K 31/506; A61K 31/437

(52) U.S. Cl. 514/253.04; 544/362; 514/318; 546/118; 546/194; 544/333; 514/256; 514/303

(57) ABSTRACT

The invention encompasses compounds of Formula I or pharmaceutically acceptable salts thereof, which are modulators of the CXCR3 chemokine receptor function useful for the treatment or prevention of pathogenic inflammatory processes, autoimmune diseases or graft rejection processes. Methods of use and pharmaceutical compositions are also encompassed.

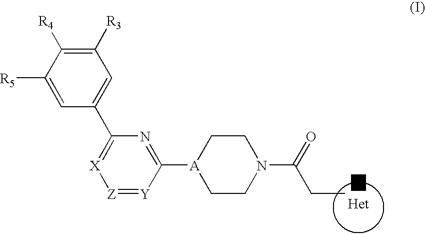

### BACKGROUND OF THE INVENTION

The chemokines are a family of small (70-120 amino acids), pro-inflammatory cytokines, with potent chemotactic activities. As their name implies, one function of chemokines, which are released by a wide variety of cells at sites of inflammation, is to attract leukocytes, including monocytes, macrophages, T lymphocytes, eosinophils, basophils and neutrophils and to promote their migration through endothelial layers. (reviewed in Schall, Cytokine, 3, 165-183 (1991) and Murphy, Rev. Immun., 12, 593-633 (1994)). In addition to their well characterized role in leukocyte trafficking, it is now also appreciated that that chemokines play a role in a number of other biological processes including cellular proliferation, hematopoiesis, angiogenesis, tumor metastasis and host defense.

These polypeptides were originally defined as having four conserved aminoterminal cysteines, and divided into two major and two minor subfamilies based on the spacing arrangement of the first cysteine pair. The two major subfamilies consist of the CXC (or α) and CC (or β) chemokines. In the CXC-chemokine family, which includes CXCL1 (MOSA or GROα), CXCL7 (NAP-2), CXCL8 (interleukin-8 or IL-8), CXCL9 (MIG), CXCL10 (IP-10) and CXCL11 (I-TAC), these two cysteines are separated by a single amino acid, while in the CC-chemokine family, which includes CCL5 (RANTES), CCL2 (monocyte chemotactic protein-1 or MCP-1), CCL8 (MCP-2), CCL7 (MCP-3), CCL3 (MIP-1α), CCL4 (MIP-1β) and CCL11 (eotaxin), these two residues are adjacent.

Some CXC-chemokines, such as CXCL1, CXCL7 and CXCL-8 are chemotactic primarily for neutrophils while another subset of CXC chemokines, including CXCL9, CXCL10 and CXCL11, are chemotactic primarily for T-lymphocytes. In comparison, the CC_chemokines, such as CCL5, CCL3, CCL4, CCL2, CCL8, CCL7 and CCL11, are more broad in their action and are chemotactic for macrophages, monocytes, T-lymphocytes, eosinophils and basophils (Deng, et al., Nature, 381, 661-666 (1996), Murphy et al. Pharmacol Revw. 52(1) 145-176, (2000).).

The chemokines bind to specific G-protein coupled receptors (GPCRs) present on leukocytes and other cells. (reviewed in Horuk, Trends Pharm. Sci., 15, 159-165 (1994), Murphy et al. Pharmacol Revw. 52(1) 145-176, (2000).) Upon interaction with their cognate ligands, chemokine receptors transduce an intracellular signal though their associated heterotrimeric G proteins, resulting in a rapid cellular responses, including an increase in intracellular calcium concentration. These chemokine receptors form a sub-family of GPCRs, which, at present, consists of a number of well characterized members with known ligands as well as a number of orphans. Unlike receptors for promiscuous classical chemoattractants such as C5a, fMLP, PAF, and LTB4, chemokine receptors are more selectively expressed on subsets of leukocytes. Thus, generation of specific chemokines provides a mechanism for recruitment of particular leukocyte subsets. The restricted expression and defined function of the chemokine receptors has focused attention on intervention in the chemokine signaling pathways as a method for highly selective intervention in pathological immunological and inflammatory processes.

Chemokine receptors, such as CCR1, CCR2A, CCR2B, CCR3, CCR4, CCR5, CXCR3, CXCR4, have been implicated as important mediators of inflammatory diseases and immunoregulatory disorders, including asthma, allergic rhinitis and atherosclerosis. They are also purported to play a role in the pathogenesis of autoimmune disorders such as rheumatoid arthritis, psoriasis, multiple sclerosis. An extensive review of the role of chemokines in disease is provided by in Seminars in Immunology., 15(1), 1-55 (2003).

A subset of chemokines are potent chemoattractants for lymphocytes. For example CXCR3 (CD183) is expressed in activated T lymphocytes, some B lymphocytes and NK cells. Expression and receptor responsiveness are both increased by activation of the T lymphocytes. The potent inflammatory cytokines CXCL10 and CXCL11 are chemoattractant for T lymphocytes and tumor infiltrating lymphocytes. The relatively restricted expression of the CXCR3 expression on these pro-inflammatory cell types mark CXCR3 as a very promising target for selective intervention in the inflammatory process. A connection with disease processes, particularly Th-1 mediated processes, is indicated by the presence of the CXCR3 on most activated T lymphocytes within inflamed joint synovium in rheumatoid arthritis as well as within inflamed tissue present in other inflammatory disorders including ulcerative colitis, Graves' disease, MS and rejecting graft tissues. (Qin, J. Clin. Invest., 101(4), 746-754 (1998), Garcia-Lopez, Lab. Investig. 81(3), 409-418 (2001), Balashov, PNAS, 96, 6873-6878 (1999), DeVries, Seminars in Immunology, 15(1), 33-48 (2003)) A similar but somewhat less pronounced association is shown with the CCR5 receptor and its ligand CCL5

Accordingly, agents which inhibit or modulate the function of chemokine receptors such as the CXCR3 receptor would be useful in treating or preventing such disorders and diseases. Data from animal models of inflammation further supports the hypothesis regarding the effectiveness of chemokine blockade, specifically CXCR3 inhibition, in diseases with clear T-lymphocyte mediated tissue damage such as transplant rejection, graft versus host disease, multiple sclerosis, optic neuritis and rheumatoid or psoriatic arthritis. Many other diseases are characterized by T lymphocyte infiltrates, and by inference are therefore also good candidates for interventions which prevent the migration of T lymphocytes. These diseases include psoriasis and other chronic inflammatory diseases of the skin such as atopic dermatitis, lichen planus and bullous pemphigoid, inflammatory bowel diseases such as ulcerative colitis and Crohn's disease and autoimmune diseases such as systemic and cutaneous lupus erythematosus, Behcet's disease, type I diabetes or Graves' disease.

Many inflammatory lung diseases such as chronic obstructive pulmonary disease, hypersensitivity pneumonitis, chronic eosinophilic pneumonia, pulmonary sarcoidosis, bronchiolitis obliterans syndrome, asthma, kidney diseases such as glomerulonephritis, pathogenesis of chronic HCV infection and atherosclerosis show a dependence on T lymphocytes and are promising targets for agents which modulate the function of chemokine receptors such as the CXCR3 receptor.

The expression of CXCR3 in some B cell tumors indicates that intervention in CXCR3 function could have beneficial effects in these cancers, particularly in suppressing metastasis.

Several methods are under investigation for modulation of chemokine receptor function. These include antibodies binding to and neutralizing the chemokine ligands, antibodies binding to and modulating the function of the chemokine receptors and small molecules which bind to and inhibit function of the chemokine receptor. The ideal method for intervention in CXCR3 mediated chemotaxis is the binding of orally bioavailable small molecules which prevent the function of the receptor. Molecules with affinity for the CXCR3 chemokine receptor and ability to modulate the function of the receptor are described here.

### SUMMARY OF THE INVENTION

The invention encompasses compounds of Formula I

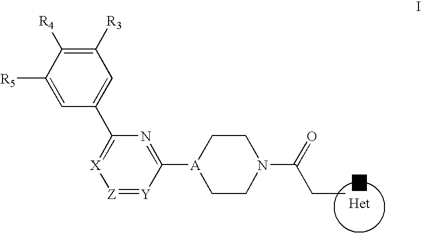

or pharmaceutically acceptable salts thereof, which are modulators of the CXCR3 chemokine receptor function useful for the treatment or prevention of pathogenic inflammatory processes, autoimmune diseases or graft rejection processes. Methods of use and pharmaceutical compositions are also encompassed.

### DETAILED DESCRIPTION OF THE INVENTION

The invention encompasses a genus of compounds of Formula I

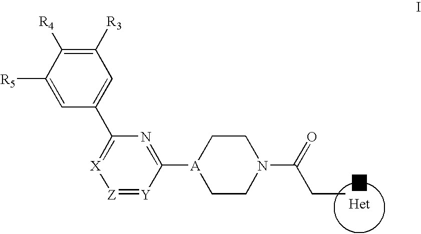

or a pharmaceutically acceptable salt thereof, wherein:

### A is CH or N;

one of X, Y and Z is N or CH, the other of X, Y and Z are CH;
R₃ is selected from the group consisting of: C₁₋₄alkyl, —CF₃, —OCF₃ and —S(O)ₙCF₃, wherein n is 0 or 2;
R₄ is selected from the group consisting of: H, halo, —OH, —OCH₃, —OCH₂CF₃ and —CF₃;
or R₃ and R₄ may be joined together with the carbon atoms to which they are attached to form a five- or six-membered monocyclic ring, said rings tetra-substituted with methyl groups as follows:

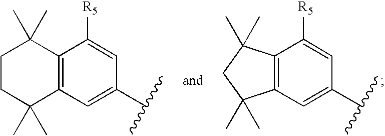

R₅ is selected from the group consisting of: C₁₋₄alkyl, C₃₋₆cycloalkyl, CF₃, —CF₂CH₃, —OCF₃ and —SCF₃; and

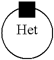

is a 5 membered non-aromatic or aromatic ring or a 9 membered fused bicyclic partially aromatic or aromatic ring, each ring containing at least 1 nitrogen atom and optionally up to 3 additional heteroatoms selected from S, O and N, said rings optionally substituted with 1 to 3 substituents independently selected from the group consisting of: oxo, hydroxy, carboxy, —CF₃, halo, —S(O)ₚ—CH₃, phenyl, C₁₋₃alkoxy and C₁₋₃alkyl, said C₁₋₃alkyl optionally substituted with carboxy or hydroxy; and
p is 0, 1 or 2.

Within this genus, the invention encompasses a sub-genus of compounds of Formula I wherein:

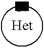

is selected from the group consisting of:

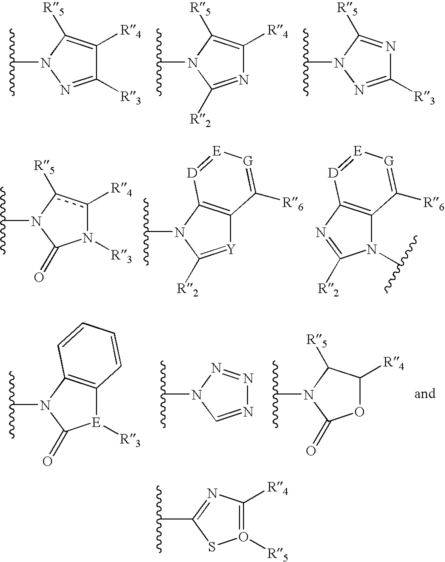

wherein D, E and G are independently C or N,
R″₂, R″₃, R″₄ and R″₅ are independently selected from the group consisting of: —H, carboxy, —CF₃, halo, methylthio, methylsulfonyl, phenyl, C₁₋₃alkoxy and C₁₋₃alkyl, said C₁₋₃alkyl optionally substituted with carboxy or hydroxy,

### R′₆ is H or OH, and

------ is an optional double bond.

Also within the genus, the invention encompasses a sub-genus of compounds of Formula I wherein A is N.

Also within the genus, the invention encompasses a sub-genus of compounds of Formula I wherein A is CH.

Within this sub-genus, the invention encompasses a class of compounds of Formula I wherein X, Y and Z are CH.

Within this sub-genus, the invention encompasses a class of compounds of Formula I wherein X is N and Y and Z are CH.

Also within this sub-genus, the invention encompasses a class of compounds of Formula I wherein Y is N and X and Z are CHI.

Also within this sub-genus, the invention encompasses a class of compounds of Formula I wherein Z is N and X and Y are CH.

In another embodiment, the invention encompasses a sub-genus of compounds of Formula I within the genus wherein R₃ and R₅ are tert-butyl.

In another embodiment, the invention encompasses a sub-genus of compounds of Formula I within the genus wherein R₃ and R₅ are CF₃.

In another embodiment, the invention encompasses a sub-genus of compounds of Formula I within the genus wherein:

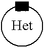

is selected from the group consisting of:

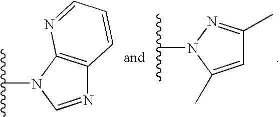

In another embodiment, the invention encompasses a sub-genus of compounds of Formula I within the genus wherein:

### A, X, Y and Z are CH;

R₃ and R₅ are tert-butyl or R₃ and R₅ are CF₃; and
R₄ is selected from H and —OCH₃.

Within this sub-genus, the invention encompasses a class of compounds of Formula I wherein:

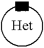

is selected from the group consisting of:

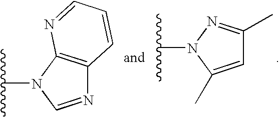

In another embodiment, the invention encompasses a compound selected from the following group:
1) 3-(2-(4-[6-(3,5-di-tert-butylphenyl)pyridine-2-yl]piperazin-1-yl)-2-oxoethyl)-3-H-imdazo[4,5-b]pyridine;
2) 3-(2-{4-[6-(3,5-di-tert-butylphenyl)-2-pyridinyl]-1-piperidinyl}-2-oxoethyl)-3H-imidazo[4,5-b]pyridine;
3) 2-(3,5-di-tert-butylphenyl)-6-{1-[(3,5-dimethyl-1-pyrazol-1H-yl)acetyl]-4-piperidinyl}pyridine;
4) 2-(3,5-di-tert-butylphenyl)-6-{1-[(3,5-dimethyl-1-1,2,4-triazol-1H-yl)acetyl]-4-piperidinyl}pyridine;
5) 3-[2-(4-{6-[3,5-bis(trifluoromethyl)phenyl]-2-pyridinyl}-1-piperidinyl)-2-oxoethyl]-3H-imidazo[4,5-b]pyridine;
6) 3-(2-{4-[6-(3,5-di-tert-butyl-4-methoxyphenyl)-2-pyridinyl]-1-piperidinyl}-2-oxoethyl)-3H-imidazo[4,5-b]pyridine;
7) 2-(3,5-di-tert-butyl-4-methoxyphenyl)-6-{1-[(3,5-dimethyl-1H-pyrazol-1-yl)acetyl]-4-piperidinyl}pyridine;
8) 2-(3,5-di-tert-butyl-4-methoxyphenyl)-6-{1-[(2,4-dimethyl-1H-imidazol-1-yl)acetyl]-4-piperidinyl}pyridine;
9) 3-[2-(4-{4-[3,5-bis(trifluoromethyl)phenyl]-2-pyrimidinyl}-1-piperidinyl)-2-oxoethyl]-3H-imidazo[4,5-b]pyridine;
10) 3-(2-{4-[2-(3,5-di-tert-butyl-4-methoxyphenyl)pyrimidin-4-yl]piperidin-1-yl}-2-oxoethyl)-3H-imidazo[4,5-b]pyridine;
11) [1-(2-{4-[2-(3,5-di-tert-butyl-4-methoxyphenyl)pyrimidin-4-yl]piperidin-1-yl}-2-oxoethyl)-5-methyl-1H-pyrazol-3-yl]acetic acid;
12) 3-(2-{4-[6-(3,5-di-tert-butylphenyl)pyrazin-2-yl]piperidin-1-yl}-2-oxoethyl)-3H-imidazo[4,5-b]pyridine;
13)-{2-[4-(3′,5′-di-tert-butylbiphenyl-3-yl)piperidin-1-yl]-2-oxoethyl}-1H-benzimidazole;
14) 3-(2-{4-[6-(3,5-di-tert-butylphenyl)-2-pyridinyl]-1-piperidinyl}-2-oxoethyl)-3H-imidazo[4,5-b]pyridine
15) 3-[2-(4-{6-[3,5-bis(trifluoromethyl)phenyl]-2-pyridinyl}-1-piperidinyl)-2-oxoethyl]-3H-imidazo[4,5-b]pyridine;
16) 2-(3,5-di-tert-butylphenyl)-6-{1-[(3,5-dimethyl-1-pyrazol-1H-yl)acetyl]-4-piperidinyl}pyridine;
17) 2-(3,5-di-tert-butylphenyl)-6-{1-[(3,5-dimethyl-1-1,2,4-triazol-1H-yl)acetyl]-4-piperidinyl}pyridine;
18) 3-(2-{4-[6-(3,5-di-tert-butyl-4-methoxyphenyl)-2-pyridinyl]-1-piperidinyl}-2-oxoethyl)-3H-imidazo[4,5-b]pyridine;
19) 2-(3,5-di-tert-butyl-4-methoxyphenyl)-6-{1-[(3,5-dimethyl-1H-pyrazol-1-yl)acetyl]-4-piperidinyl}pyridine;
20) 2-(3,5-di-tert-butyl-4-methoxyphenyl)-6-{1-[(2,4-dimethyl-1H-imidazol-1-yl)acetyl]-4-piperidinyl}pyridine;
21) 3-[2-(4-{4-[3,5-bis(trifluoromethyl)phenyl]-2-pyrimidinyl}-1-piperidinyl)-2-oxoethyl]-3H-imidazo[4,5-b]pyridine; and
22) 3-(2-{4-[6-(3,5-di-tert-butylphenyl)-2-pyridinyl]-1-piperazinyl}-2-oxoethyl)-3H-imidazo[4,5-b]pyridine,
   or a pharmaceutically acceptable salt of any of the aforementioned.

The invention also encompasses a pharmaceutical composition comprising a compound of Formula I in combination with a pharmaceutically acceptable carrier.

The invention also encompasses a method for treating a disease or condition mediated by the CXCR3 chemokine receptor comprising administering to a patient in need of such treatment a therapeutically effective amount of a compound of Formula I.

The invention also encompasses a method for treating a disease or condition mediated by the CXCR3 chemokine receptor comprising administering to a patient in need of such treatment a therapeutically effective amount of a compound of Formula I, wherein the disease or condition is selected from the group consisting of: acute and chronic transplant rejection, psoriasis, rheumatoid arthritis and multiple sclerosis.

The term “halogen” or “halo” includes F, Cl, Br, and I.

The term “alkyl” means linear or branched structures and combinations thereof, having the indicated number of carbon atoms. Thus, for example, C₁₋₆alkyl includes methyl, ethyl, propyl, 2-propyl, s- and t-butyl, butyl, pentyl, hexyl and 1,1-dimethylethyl.

The term “cycloalkyl” means mono-, bi- or tri-cyclic structures, optionally combined with linear or branched structures, having the indicated number of carbon atoms. Examples of cycloalkyl groups include cyclopropyl, cyclopentyl, cycloheptyl, adamantyl, cyclododecylmethyl, 2-ethyl-1-bicyclo[4.4.0]decyl, cyclobutylmethyl, cyclopropylmethyl 1-methylcyclopropyl and the like.

### Optical Isomers-Diastereomers-Geometric Isomers-Tautomers

Some of the compounds described herein may exists as mixtures of tautomers. The term “tautomers” embraces the standard meaning of the term, i.e. a type of isomerism in which two or more isomers are rapidly interconverted so that they ordinarily exist together in equilibrium. Tautomers include, e.g., compounds that undergo facile proton shifts from one atom of the compound to another atom of the compound. Some of the compounds described herein may exist as tautomers with different points of attachment of hydrogen. Such an example might be a ketone and its enol form known as keto-enol tautomers or an amide and its hydroxy imine tautomer. The individual tautomers of the compounds of Formula I, as well as mixtures thereof, are included in the scope of this invention. By way of illustration, tautomers included in this definition include, but are not limited to:

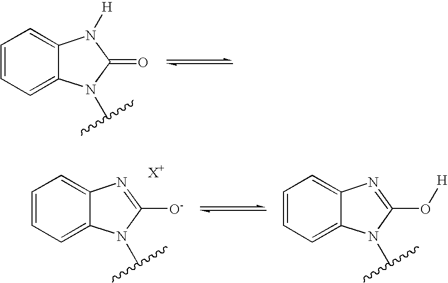

### Salts

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds of Formula I are meant to also include the pharmaceutically acceptable salts.

### Utilities

The compounds of the present invention are modulators of CXCR3 chemokine receptor function and are of use in antagonizing chemokine mediated cell signalling and in particular are of use in the prophylaxis and/or treatment of diseases or disorders involving inappropriate T-cell trafficking. The invention extends to such a use and to the use of the compounds of Formula I for the manufacture of a medicament for treating such diseases and disorders. Particular diseases include inflammatory, autoimmune and immunoregulatory disorders.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

Diseases or conditions of humans or other species which can be treated with compounds of Formula I, include, but are not limited to: autoimmune mediated inflammatory or allergic diseases and conditions, including respiratory diseases such as asthma, particularly bronchial asthma, systemic lupus erythematosus, ankylosing spondylitis, systemic sclerosis, autoimmune diseases, such as rheumatoid arthritis, psoriatic arthritis, multiple sclerosis, systemic lupus erythematosus, myasthenia gravis, juvenile onset diabetes; glomerulonephritis, autoimmune thyroiditis, Behcet's disease; acute and chronic graft rejection (e.g., in transplantation), including allograft rejection or graft-versus-host disease; inflammatory bowel diseases, such as Crohn's disease and ulcerative colitis; spondyloarthropathies; scleroderma; psoriasis (including T-cell mediated psoriasis); vasculitis (e.g., necrotizing, cutaneous, and hypersensitivity vasculitis); cancers with leukocyte infiltration of the skin or organs. Other diseases or conditions in which undesirable inflammatory responses are to be inhibited can be treated, including, but not limited to, reperfusion injury, atherosclerosis, certain hematologic malignancies, and polymyositis.

The compounds of the present invention are accordingly useful in treating, preventing, ameliorating, controlling or reducing the risk of a wide variety of inflammatory and immunoregulatory disorders and diseases as well as autoimmune pathologies. In a specific embodiment, the present invention is directed to the use of the subject compounds for treating, preventing, ameliorating, controlling or reducing the risk of autoimmune diseases, such as rheumatoid arthritis, multiple sclerosis, psoriasis or psoriatic arthritis.

In another aspect, the instant invention may be used to evaluate putative specific agonists or antagonists of chemokine receptors, including CXCR3. Accordingly, the present invention is directed to the use of these compounds in the preparation and execution of screening assays for compounds which modulate the activity of chemokine receptors. For example, the compounds of this invention are useful for isolating receptor mutants, which are excellent screening tools for more potent compounds. Furthermore, the compounds of this invention are useful in establishing or determining the binding site of other compounds to chemokine receptors, e.g., by competitive inhibition. The compounds of the instant invention are also useful for the evaluation of putative specific modulators of the chemokine receptors, including CXCR3. As appreciated in the art, thorough evaluation of specific agonists and antagonists of the above chemokine receptors has been hampered by the lack of availability of non-peptidyl (metabolically resistant) compounds with high binding affinity for these receptors. Thus the compounds of this invention are commercial products to be sold for these purposes.

The present invention is further directed to a method for the manufacture of a medicament for treating CXCR3 mediated diseases in humans and animals comprising combining a compound of the present invention with a pharmaceutical carrier or diluent.

In a preferred aspect of the present invention, a subject compound may be used in a method of inhibiting the binding of a chemokine to a chemokine receptor, such as CXCR3, of a target cell, which comprises contacting the target cell with an amount of the compound which is effective at inhibiting the binding of the chemokine to the chemokine receptor.

The subject treated in the methods above is a mammal, preferably a human being, male or female, in whom modulation of chemokine receptor activity is desired. “Modulation” as used herein is intended to encompass antagonism, agonism, partial antagonism, inverse agonism and/or partial agonism. In a preferred aspect of the present invention, modulation refers to antagonism of chemokine receptor activity. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention to the individual in need of treatment.

As used herein, the term “treatment” refers both to the treatment and to the prevention or prophylactic therapy of the aforementioned conditions.

### Dose Ranges

The magnitude of prophylactic or therapeutic dose of a compound of Formula I will, of course, vary with the nature and severity of the condition to be treated, and with the particular compound of Formula I used and its route of administration. The dose will also vary according to the age, weight and response of the individual patient. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases.

For use where a composition for intravenous administration is employed, a suitable dosage range is from about 0.01 mg to about 25 mg (preferably from 0.1 mg to about 10 mg) of a compound of Formula I per kg of body weight per day.

In the case where an oral composition is employed, a suitable dosage range is, e.g. from about 0.01 mg to about 100 mg of a compound of Formula I per kg of body weight per day, preferably from about 0.1 mg to about 10 mg per kg.

For use where a composition for sublingual administration is employed, a suitable dosage range is from 0.01 mg to about 25 mg (preferably from 0.1 mg to about 5 mg) of a compound of Formula I per kg of body weight per day.

### Pharmaceutical Compositions

Another aspect of the present invention provides pharmaceutical compositions which comprises a compound of Formula I and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, additional active ingredient(s), and pharmaceutically acceptable excipients.

Any suitable route of administration may be employed for providing a mammal, especially a human with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a compound of Formula I as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

The compositions include compositions suitable for oral, sublingual, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the compounds of the present invention are conveniently delivered in the form of an aerosol spray presentation from pressurized packs or nebulizers.

The compounds may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of a compound of Formula I in suitable propellants, such as fluorocarbons or hydrocarbons and dry powder inhalation (DPI) aerosol, which may be formulated as a dry powder of a compound of Formula I with or without additional excipients.

Suitable topical formulations of a compound of formula I include transdermal devices, aerosols, creams, ointments, lotions, dusting powders, and the like.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or non-aqueous techniques.

In addition to the common dosage forms set out above, the compounds of Formula I may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet contains from about 1 mg to about 500 mg of the active ingredient and each cachet or capsule contains from about 1 to about 500 mg of the active ingredient.

### Combination Therapy

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I. Examples of other active ingredients that may be combined with a compound of Formula I, either administered separately or in the same pharmaceutical compositions, include, but are not limited to: (a) VLA-4 antagonists such as those described in U.S. Pat. No. 5,510,332, WO97/03094, WO97/02289, WO96/40781, WO96/22966, WO96/20216, WO96/01644, WO96/06108, WO95/15973 and WO96/31206, as well as natalizumab; (b) steroids such as beclomethasone, methylprednisolone, betamethasone, prednisone, dexamethasone, and hydrocortisone; (c) immunosuppressants such as cyclosporin, tacrolimus, rapamycin and other FK-506 type immunosuppressants; (d) immunomodulatory antibody therapies including anti-TNF therapies such as Etanercept (Enbrel®), Infliximab (Remicade®), Adalimumab (Humira®) or other TNF peptide or receptor sequestrants; Efalizumab (Raptiva®), Daclizumab (Zenapax®), Basiliximab (Simulect®), Rituximab (Rituxan®), visilizumab (Nuvion®), Abatacept (Orencia®) or other interleukin peptide or receptor binding antibodies; (e) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (f) non-steroidal anti-asthmatics such as β2-agonists (terbutaline, metaproterenol, fenoterol, isoetharine, albuterol, bitolterol, salmeterol and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (g) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac and zidometacin), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine, olsalazine, mesalamine and balsalazide) and the pyrazolones (apazone, bezpiperylon, feprazone, mofebutazone, oxyphenbutazone, phenylbutazone); (h) cyclooxygenase-2 (COX-2) inhibitors such as celecoxib, rofecoxib, and parecoxib; (i) inhibitors of phosphodiesterase type IV (PDE-IV); (j) antagonists of the other chemokine receptors, especially CCR1, CCR2, CCR5 and CCR3; (k) cholesterol lowering agents such as HMG-CoA reductase inhibitors (lovastatin, simvastatin, pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; (l) anti-diabetic agents such as insulin, sulfonylureas, biguanides (metformin), a-glucosidase inhibitors (acarbose), glitazars (muraglitazar) and glitazones (troglitazone, pioglitazone, englitazone, MCC-555, BRL49653 and the like); (m) preparations of interferon beta (Avonex®, Rebif®, interferon beta-1a, Betaseron®, interferon beta-1b); (n) anticholinergic agents such as muscarinic antagonists (ipratropium and tiatropium); (o) current treatments for multiple sclerosis, including prednisolone, glatiramer, deoxyadenosine, mitoxantrone, methotrexate, and cyclophosphamide; (p) p38 kinase inhibitors; (q) DMARDs, such as methotrexate, leflunamide or plaquenil; (r) other compounds such as 5-aminosalicylic acid and prodrugs thereof, antimetabolites such as azathioprine, mycophenolate and 6-mercaptopurine, cytotoxic cancer chemotherapeutic agents and cytokine sequestrants.

The weight ratio of the compound of the Formula I to the second active ingredient may be varied and will depend upon the effective dose of each ingredient. Generally, an effective dose of each will be used. Thus, for example, when a compound of the Formula I is combined with an NSAID the weight ratio of the compound of the Formula I to the NSAID will generally range from about 1000:1 to about 1:1000, preferably about 200:1 to about 1:200. Combinations of a compound of the Formula I and other active ingredients will generally also be within the aforementioned range, but in each case, an effective dose of each active ingredient should be used.

### Methods of Synthesis

The following abbreviations are used in the synthetic schemes:

Ac is acetyl [CH₃C(O)—]; Ac₂O is acetic anhydride; 9-BBN is 9-borabicyclo[3.3.1]nonane; Bn is benzyl; BOC is tert Butyloxycarbonyl; DIAD is diisopropylazodicarboxylate; DIBAL is diisobutylaluminum hydride; DMF is N,N-dimethylformamide; DMSO is dimethyl sulfoxide; EDAC (or EDC) is 1-ethyl-3-[3-(dimethylamino)propyl]-carbodiimide HCl; Et₃N is triethylamine; Et is ethyl; EtOAc is ethyl acetate; EtOH is ethanol; HCl is hydrochloric acid; HOBt is 1-hydroxybenzotriazole; HPLC is high performance liquid chromatography; LG is leaving group; M is molar; mmol is millimole; Me is methyl; MeOH is methanol; MsCl methanesulfonyl chloride; N is normal; NaHMDS is sodium hexamethyldisiliazide; NaOAc is sodium acetate; NaOtBu is sodium tert-butoxide; NMO is N-methylmorpholine N oxide; PG is protecting group; Pd(dba)₂ is bis(dibenzylideneacetone) palladium; PdCl₂(Ph₃P)₂ is dichlorobis-(triphenylphosphene)palladium; Ph is phenyl; PhMe is toluene; PPh₃ is triphenylphosphine; PMB is para-methoxybenzyl; RT is room temperature; TBAF is tetrabutyl ammonium fluoride; TBS is tert-butyldimethylsilyl; tBu is tert-butyl; Tf is triflate; TFA is trifluoroacetic acid; THF is tetrahydrofuran; TLC is thin layer chromatography; TMS is trimethylsilyl; TPAP is tetrapropylammonium perruthenate;

### General Schemes

The substituted pyridine, pyrimidine or pyrazine compounds of this invention can be prepared by any of several known methods. The specific examples detailed below employ some of the following general procedures.

Trisubstituted aryl and heteroaryl intermediates 1 may be commercially available or may be prepared from readily accessible anilines, phenols or other simpler congeners via a host of routes which will be obvious to a practicing synthetic chemist.

The elaborated substituted biaryl piperidines 9 are accessible from these intermediates as shown in Scheme 1, 2 or alternate synthetic pathways as reported in the literature. Various aryl coupling methods are well suited to production of these intermediates. Typical examples of this very general method as depicted in step (d) are reported in [Kotha, Lahiri, Kashinath Tetrahedron, 58, 9633-9695, 2002; Tyrrell, Brookes Synthesis 2003, 4, 469-483.] The variation of the Suzuki coupling illustrated in Scheme 1 is used for synthesis of many of the analogs reported here. The tetrahydropyridine partners such as 7 are easily prepared from commercially available or readily accessible ketones as shown.

Deprotection and coupling with a heteroarylacetate yields the completed analogs 10. Some analogs will contain functionality which will require a final deprotection step. In many cases this constitutes an ester hydrolysis under typical conditions. Some complex substitution patterns in heteroaryl intermediates are most readily accessible by de novo ring synthesis as outlined in Schemes 2 and 3.

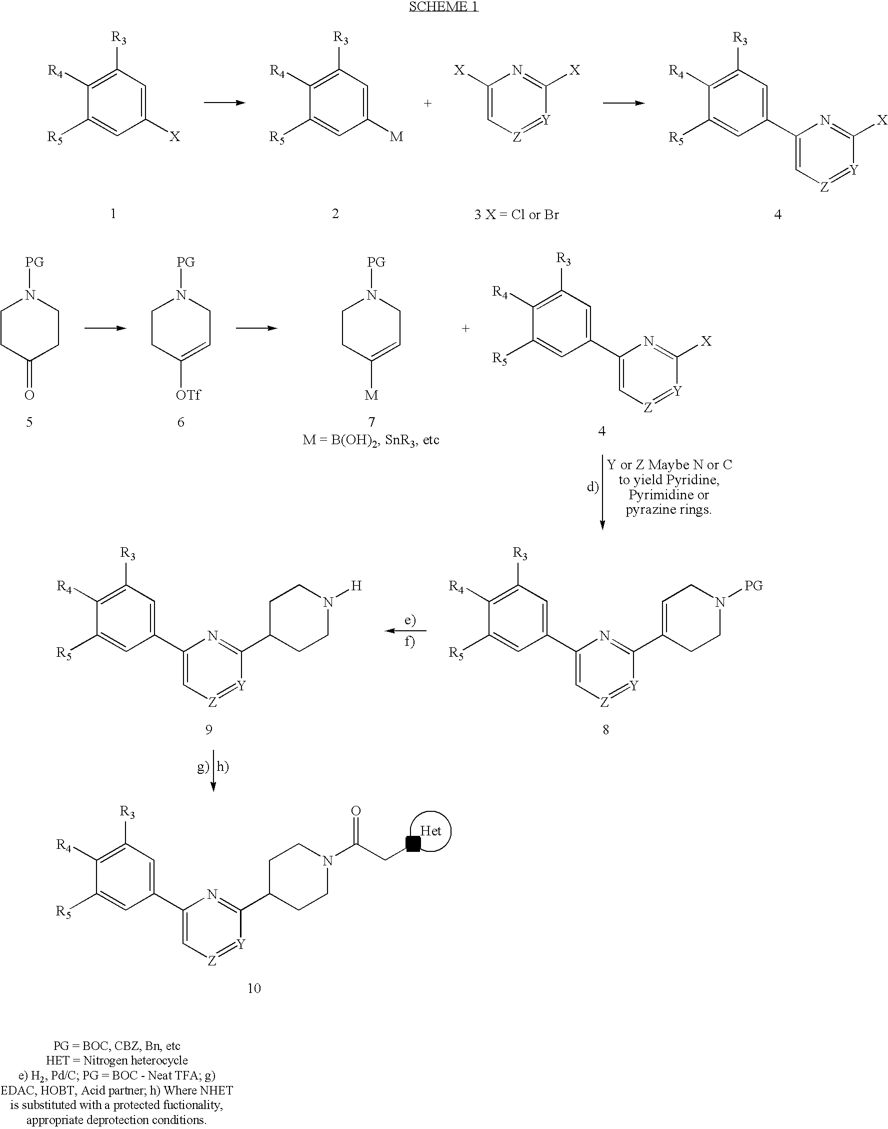

A typical example of de a de novo ring synthesis is shown in Scheme 2 for the 2-arylpyrimidine case. Each of these examples will follow a different route and the required route will be clear to an experienced synthetic chemist. A good review of the state of the art is contained in Katritzky, Alan R. (1984) Comprehensive heterocyclic chemistry: the structure, reactions, synthesis and uses of heterocyclic compounds, Oxford; Pergamon, New York.

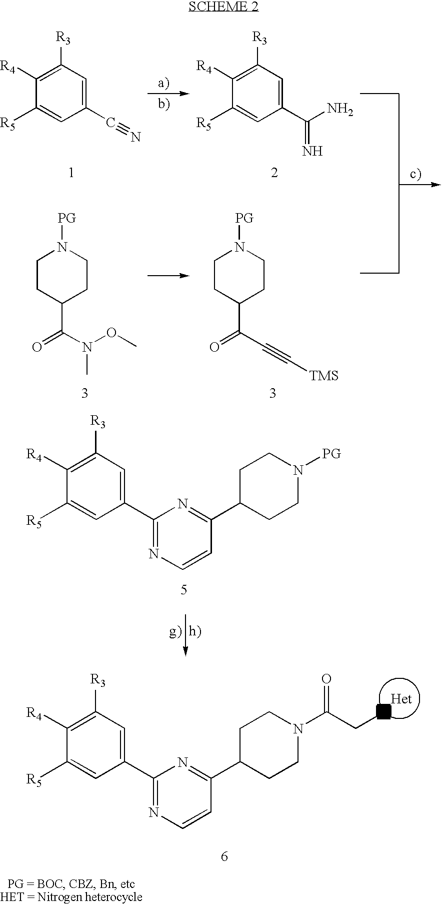

In many of the current examples, additional polysubstituted heterocyclic fragments will be required. Several examples derive from substituted pyrazoles, thiazoles and imidazopyridines. The methods of synthesis will be well known to a practicing synthetic chemist and are summarized in Katritzky, cited above. In addition to the various published routes to these intermediates, the general procedures of Scheme 3 have been found to give access to the desired intermediate heteroaryls. In Scheme 3, R and R″ are alkyl or aryl substituents as desired, and the ester residue E can also be alkyl or benzyl as needed to allow selective deprotection.

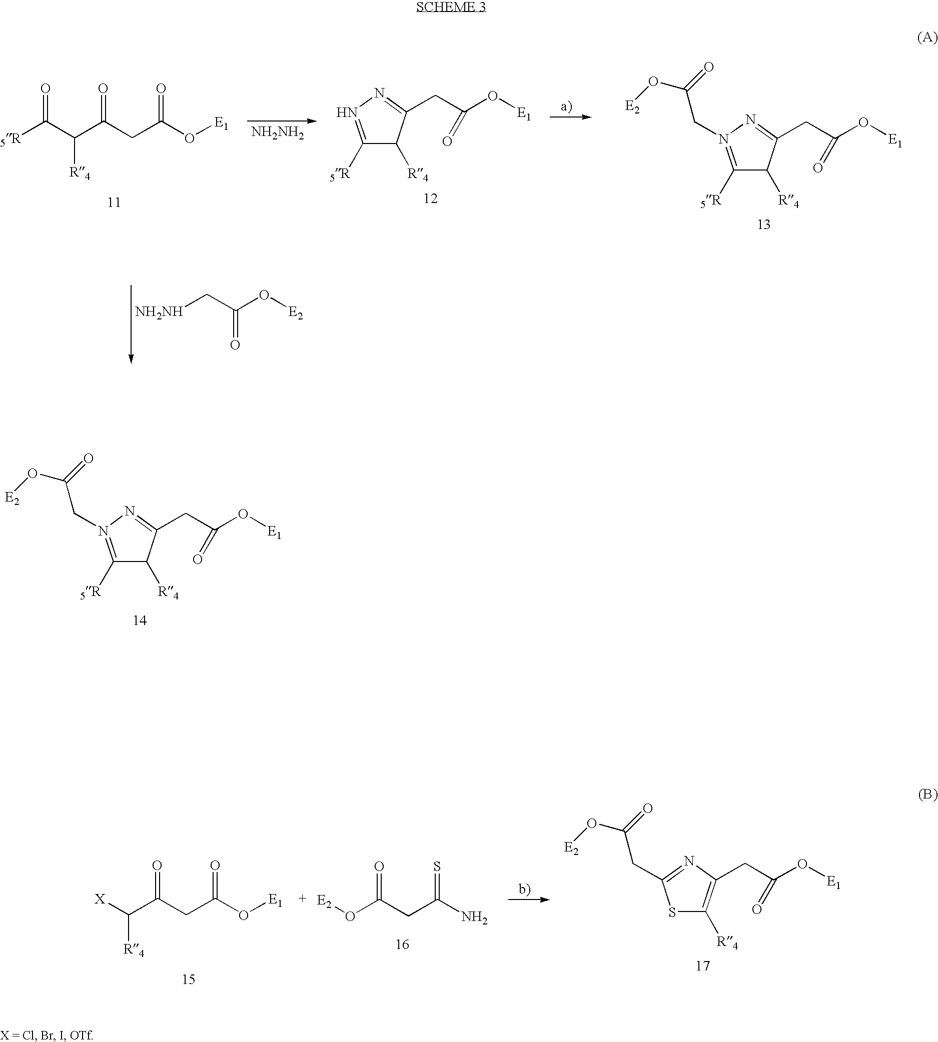

### EXAMPLE 1

### Preparation of 3-(2-(4-[6-(3,5-di-tert-butylphenyl)pyridine-2-yl]piperazin-1-yl)-2-oxoethyl)-3-H-imdazo[4,5-h]pyridine

### Step 1 2-Bromo-6-(3,5-di-tert-butylphenyl)pyridine

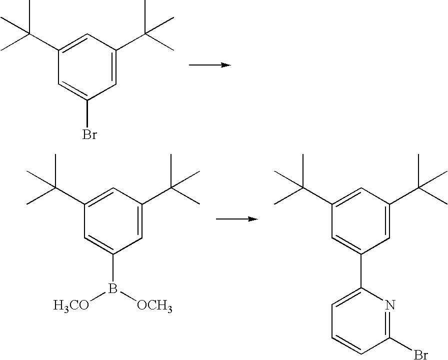

A −78° solution of 3,5-di-tert-butyl-bromobenzene (1.462 g; 5.43 mmol) in dry THF (15 mL) was treated with a solution of n-butyllithium (5.02 mL; 1.19M in hexanes; 5.97 mmol). The solution was stirred at −78° for 15 min, then treated with trimethylborate (740 μL; 6.52 mmol). The solution was allowed to warm to ambient temperature and evaporated to a residue which was used without further purification.

A solution of the crude dimethyl (3,5-di-tert-butylphenyl)boronate prepared as described above in dimethoxyethane (10 mL) was treated with 2,6-dibromopyridine (1.158 g; 4.89 mmol). After dissolution was complete tetrakis(triphenylphosphine)palladium(0) (282 mg; 0.244 mmol) was added, followed by aq. sodium carbonate (30 mL; 2M). The mixture was refluxed 5 h. The reaction was partitioned between isopropyl acetate and water. The organic was dried over MgSO₄, filtered and evaporated to a residue. The crude product was chromatographed over silica gel (5% to 50% MTBE/hexane; linear gradient). The fractions containing the title compound were combined and rechromatographed over silica gel (1:1 hexane/CH₂Cl₂) to afford the pure title compound (460 mg).

500 MHz NMR (CDCl₃): δ 7.81 (d, 2H, J=1.8 Hz), 7.69 (d, 1H, J=7.1 Hz), 7.61 (t, 1H, J=7.7 Hz), 7.55 (t, 1H, J=1.9 Hz), 7.42 (d, 1H, J=7.4 Hz). LRMS calc: 345.1 obs: 346.1 (M+H).

### Step 2. 1-[6-(3,5-di-tert-butylphenyl)pyridine-2-yl]piperazine

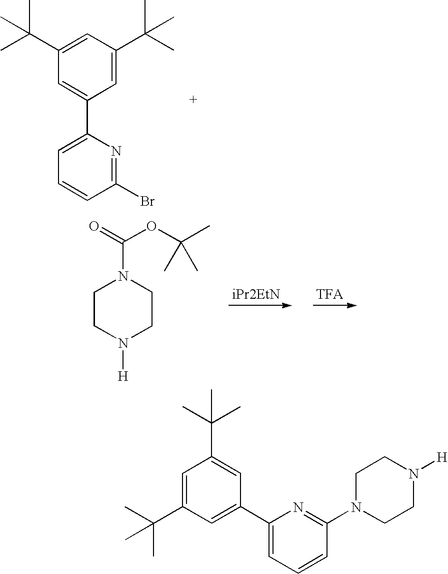

A solution of tert-butyl piperazine-1-carboxylate (614 mg; 3.30 mmol) in DMF (5 mL) was treated sequentially with 2-bromo-6-(3,5-di-tert-butylphenyl)pyridine (460 mg; 1.33 mmol) and diisopropylethylamine (0.348 mL; 2.0 mmol). The reaction was stirred at 120° C. for 24 hours. The reaction was partitioned between isopropyl acetate (15 mL) and pH 7 phosphate buffer solution (15 mL). The organic was washed twice more with pH 7 phosphate buffer solution (2×15 mL), then dried over MgSO₄, filtered and concentrated to a residue. The crude product was chromatographed on silica gel and eluted with straight dichloromethane. The title compound (92.0 mg) was isolated.

500 MHz NMR (CD₃OD): δ 7.84 (d, 2H, J=1.8 Hz), 7.62 (t, 1H, J=7.9 Hz), 7.49 (t, 1H, J=1.7 Hz), 7.16 (d, 1H, J=7.3 Hz), 6.75 (d, 1H, J=8.5 Hz), 3.62 (bt, 4H, J=6.0 Hz), 3.58 (bs, 4H), 1.49 (s, 9H), 1.38 (s, 18H).

A solution of tert-butyl 4-[6-(3,5-di-tert-butylphenyl)pyridin-2-yl]piperazine-1-carboxylate (92.0 mg; 0.204 mmol) in dichloromethane (2 mL) was treated with trifluoroacetic acid (0.5 mL). The reaction was stirred 8 h and flushed with toluene (3×2 mL). The reaction was partitioned between saturated sodium bicarbonate solution (5 mL) and isopropyl acetate (5 mL). The organic was dried over MgSO₄, filtered and concentrated to a residue to yield 54.0 mg of the title compound.

500 MHz NMR (CD₃OD): δ 7.84 (d, 2H, J=1.8 Hz), 7.62 (t, 1H, J=8.0 Hz), 7.48 (t, 1H, J=1.8 Hz), 7.15 (d, 1H, J=7.5 Hz), 6.74 (d, 1H, J=8.2 Hz), 3.62 (t, 4H, J=5.1 Hz), 2.98 (t, 4H, J=5.2 Hz), 1.38 (s, 18H). LRMS calc: 351.3 obs: 352.4 (M+H).

### Step 3. 3H-imidazo[4,5-b]pyridine 3-ylacetic acid

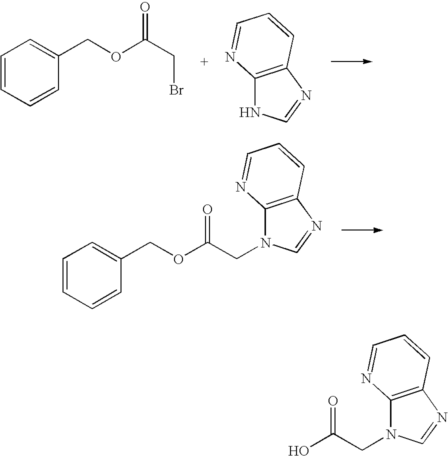

A solution of 3H-imidazo[4,5-b]pyridine (5.03 g; 42.2 mmol,) and benzyl bromoacetate (6.68 mL; 42.2 mmol) in DMF (125 mL) was treated with cesium carbonate (27.5 g; 84.4 mmol) and stirred 8 h. The reaction was partitioned between pH 4 phthalate buffer solution (250 mL) and isopropyl acetate (250 mL) and then washed twice with water (2×150 mL). The organic was dried over MgSO₄, filtered and concentrated to a residue (3.8 g; 14.2 mmol).

A solution of benzyl 3H-imidazo[4,5-b]pyridine-3-ylacetate (3.8 g; 14.2 mmol) in ethyl acetate (60 mL), was treated with 10% Pd on carbon catalyst (750 mg). The hydrogenation was carried out at 50 psi for 8 h and the reaction was filtered through Celite. Evaporation of the filtrate afforded the title compound.

### Step 4. 3-(2-(4-[6-(3,5-di-tert-butylphenyl)pyridine-2-yl]piperazin-1-yl)-2-oxoethyl)-3-H-imdazo[4,5-b]pyridine

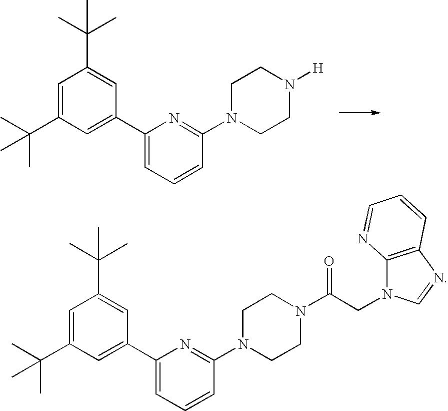

A solution of 3H-imidazo[4,5-b]pyridine-3-ylacetic acid (59.3 mg; 0.307 mmol) in 1.0 mL DMF, was treated sequentially with 4-methylmorpholine (0.051 mL; 0.461 mmol), 1-hydroxybenzotriazole hydrate (62.3 mg; 0.461 mmol), EDC (88.4 mg; 0.461 mmol) and 1-[6-(3,5-di-tert-butylphenyl)pyridine-2-yl]piperazine (54.0 mg; 0.154 mmol) and stirred at ambient temperature for 8 h. The reaction was partitioned between isopropyl acetate (10 mL) and saturated sodium bicarbonate solution (15 mL). The organic was washed twice more with aqueous sodium bicarbonate (2×15 mL). The organic was then dried over MgSO₄, filtered and evaporated to a residue. The crude product was chromatographed on silica gel and eluted with straight dichloromethane. The title compound (65.0 mg) was isolated.

500 MHz NMR (CDCl₃): δ 8.39 (dd, 1H, J=4.8, 1.4 Hz), 8.37 (s, 1H), 8.12 (dd, 1H, J=8.0, 1.3 Hz), 7.87 (d, 1H, J=1.8 Hz), 7.66 (t, 1H, J=8 Hz), 7.5 (t, 1H, J=3.4 Hz), 7.37 (dd, 1H, J=8, 4.8 Hz), 7.21 (d, 1H, J=7.5 Hz), 6.80 (d, 1H, J=8.4 Hz), 5.43 (s, 2H), 3.89 (s, 4H), 3.77 (q, 2H, J=3.4 Hz), 3.7 (q, 2H, J=3.3 Hz), 1.39 (s, 18H).

### EXAMPLE 2

### Preparation of 3-(2-{4-[6-(3,5-di-tert-butylphenyl)-2-pyridinyl]-1-piperidinyl}-2-oxoethyl)-3H-imidazo[4,5-b]pyridine

### Step 1. tert-butyl-6-(3,5-di-tert-butylphenyl)-3′,6′-dihydro-2,4′-bipyridine-1′(2′H)-carboxylate

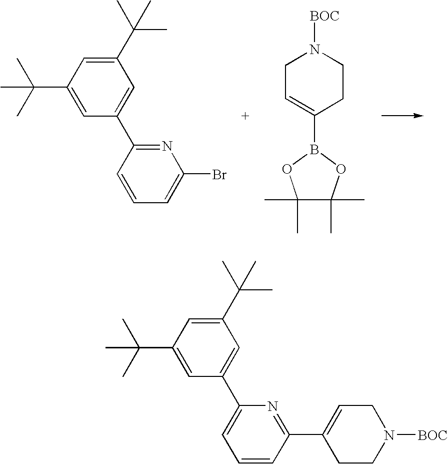

A solution of 2-bromo-6-(3,5-di-tert-butylphenyl)pyridine (EXAMPLE 1 Step 1. 732 mg; 2.114 mmol) in DME (6 mL) was treated with tert-butyl-4-(4,4,5,5-tetramethyl-1,3,2-dioxaborolan-2-yl)-3,6-dihydro-1(2H)-pyridinecarboxylate (980 mg; 3.171 mmol; prepared using the method of Eastman, P. R. Tet. Lett. 2000, 41 (19), 3705). Tetrakis(triphenylphosphine)palladium (0) (244 mg; 0.211 mmol) was added, followed by aq. Na₂CO₃ (15 mL, 2M). The mixture was refluxed for 5 h. The mixture was partitioned between iPrOAc and water. The organic was dried over MgSO₄, filtered and evaporated to a solid. The solid was chromatographed (prep. TLC; CH₂Cl₂). The major product band was recovered and rechromatographed (prep. TLC; 2:1 hex/MTBE). The major band was recovered to give the title compound (174 mg).

500 MHz NMR (CDCl₃): δ 7.89 (d, 2H, J=1.8 Hz), 7.73 (t, 1H, J=7.9 Hz), 7.62 (d, 1H, J=7.8 Hz), 7.53 (t, 1H, J=1.9 Hz), 7.34 (bd, 11, J=7.8 Hz), 6.78 (vbs, 1H), 4.20 (bd, 2H, J=3.0 Hz), 3.72 (vbs, 2H), 2.79 (vbs, 2H), 1.53 (s, 9H), 1.43 (s, 18H). LRMS calc: 448.3 obs: 449.2 (M+H).

### Step 2. 4-[6-(3,5-di-tert-butylphenyl)-2-pyridinyl]-1-piperidine bis(hydrotrifluoroacetate)

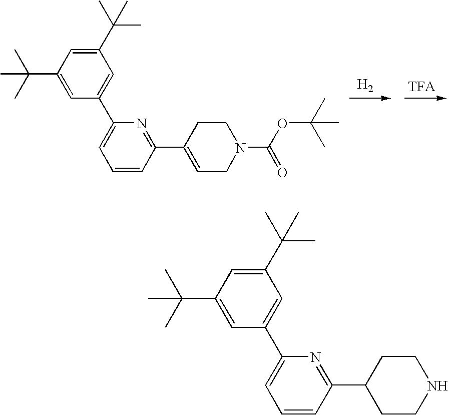

A solution of tert-butyl-6-(3,5-di-tert-butylphenyl)-3′,6′-dihydro-2,4′-bipyridine-1′(2′H)-carboxylate (174 mg; 0.388 mmol) in ethanol (6 mL) was treated with 10% Pd/C hydrogenation catalyst (175 mg). The mixture was shaken under a hydrogen atmosphere (50 psi) for 1.5 h. The mixture was filtered through Celite and evaporated to give the title compound (148 mg).

500 MHz NMR (CDCl₃): δ 7.82 (d, 2H, J=1.9 Hz), 7.65-7.55 (vbm, 3H), 7.26-7.18 (vbs, 1H), 4.24 (vbs, 2H), 2.97 (vbs, 3H), 2.09 (vbs, 2H), 1.79 (vbs, 2H), 1.54 (s, 9H), 1.40 (s, 18H). LRMS calc: 450.3 obs: 451.3 (M+H).

A solution of tert-butyl-4-[6-(3,5-di-tert-butylphenyl)-2-pyridinyl]-1-piperidinecarboxylate (148 mg; 0.329 mmol) in dry CH₂Cl₂ (5 mL) was treated with CF₃CO₂H (2 ml). The solution was stirred at ambient, temperature for 2 h. The solution was diluted with toluene (5 mL) and evaporated to a residue. The operation was repeated, affording the title compound (150 mg).

500 MHz NMR (CD3OD): δ 8.49 (t, 1H, J=8.0 Hz), 8.08 (d, 1H, J=8.0 Hz), 7.87 (d, 1H, J=7.8 Hz), 7.77 (t, 1H, J=1.7 Hz), 7.74 (d, 2H, J=1.8 Hz), 3.62-3.54 (m, 3H), 3.19 (dt, 2H, Jₜ=12.8, J_{d}=2.3 Hz), 2.31 (bd, 2H, J=14.2 Hz), 2.17 (dquart, 2H, J_{q}=13.5, J_{d}=3.5 Hz), 1.41 (s, 18H). LRMS calc: 350.3 obs: 351.4 (M+H).

### Step 3. 3-(2-{4-[6-(3,5-di-tert-butylphenyl)-2-pyridinyl]-1-piperidinyl}-2-oxoethyl)-3H-imidazo[4,5-b]pyridine

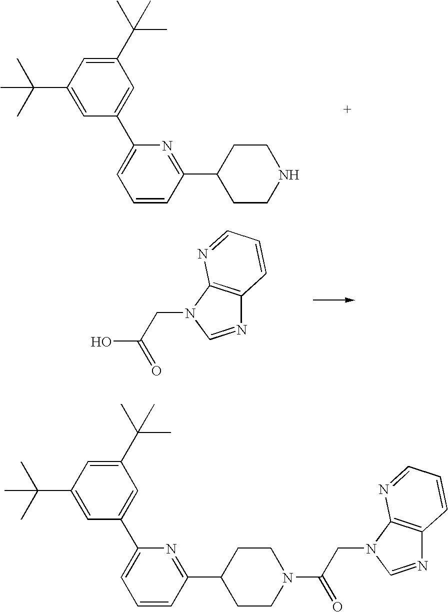

A solution of 2-(3,5-di-tert-butylphenyl)-6-(4-piperidinyl)pyridine (34 mg; 0.097 mmol) in DMF (1 mL) was treated sequentially with 3H-imidazo[4,5-b]pyridin-3-ylacetic acid (34 mg; 0.194 mmol), N-methylmorpholine (27 μL; 0.243 mmol), HOBt (33 mg; 0.243 mmol) and EDC (47 mg; 0.243 mmol). The mixture was stirred at ambient temperature for 2 h. The reaction was partitioned between iPrOAc and aq. NaHCO₃. The organic was dried, filtered and evaporated to a residue. The residue was chromatographed over silica gel (prep. TLC; 20:1 CH₂Cl₂/MeOH). The most polar mobile band was recovered, affording the title compound (30 mg).

500 MHz NMR (CDCl₃): δ 8.40 (d, 1H, J=4.6 Hz), 8.26 (bs, 1H), 8.12 (bd, 1H, J=7.8 Hz), 7.83 (d, 2H, J=1.9 Hz), 7.72 (t, 1H, J=7.8 Hz), 7.59 (d, 1H, J=7.8 Hz), 7.53 (t, 1H, J=1.7 Hz), 7.27 (dd, 1H, J=7.3, 4.8 Hz), 7.10 (d, 1H, J=7.8 Hz), 5.26 (½AB, 1H, J=16.5 Hz), 5.22 (½AB, 1H, J=16.4 Hz), 4.73 (bd, 1H, J=13.5 Hz), 4.18 (bd, 1H, J=13.3 Hz), 3.42 (bt, 1H, J=12.7 Hz), 3.11 (tt, 1H, J=11.5, 3.6 Hz), 2.94 (bt, 1H, J=12.7 Hz), 2.22 (bd, 1H, J=12.5 Hz), 2.13 (bd, 1H, J=13.7 Hz), 1.95 (dhex, 2H, Jₕ=12.3, J_{d}=3.5 Hz), 1.42 (s, 18H). LRMS calc: 509.3 obs: 510.3 (M+H).

### EXAMPLE 3

### Preparation of 2-(3,5-di-tert-butylphenyl)-6-{1-[(3,5-dimethyl-1-pyrazol-1H-yl)acetyl]-4-piperidinyl}pyridine

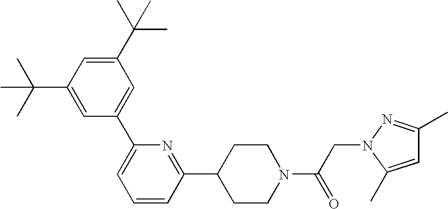

Using the method of Example 2, Step 3 with the product of Example 2, Step 2 and (3,5-dimethyl-1H-pyrazol-1-yl)acetic acid as starting materials the title compound was obtained.

500 MHz NMR (CDCl₃): δ 7.85 (d, 2H, J=1.6 Hz), 7.72 (t, 1H, J=7.8 Hz), 7.60 (d, 1H, J=7.8 Hz), 7.54 (bs, 1H), 7.10 (d, 1H, J=7.8 Hz), 5.90 (bs, 1H), 4.97 (½AB, 1H, J=16.2 Hz), 4.92 (½AB, 1H, J=16.2 Hz), 4.74 (bd, 1H, J=13.3 Hz), 4.14 (bd, 1H, J=13.2 Hz), 3.30 (bt, 1H, J=11.9 Hz), 3.07 (tt, 1H, J=11.7, 3.4 Hz), 2.88 (dt, 1H, Jₜ=13.0, J=2.3 Hz), 2.29 (s, 3H), 2.26 (s, 3H), 2.13 (bt, 1H, J=14.3 Hz), 1.92-1.80 (m, 2H), 1.44 (s, 18H). LRMS calc: 486.3 obs: 487.4 (M+H).

### EXAMPLE 4

### Preparation of 2-(3,5-di-tert-butylphenyl-6-{1-[(3,5-dimethyl-1-1,2,4-triazol-1H-yl)acetyl]-4-piperidinyl}pyridine

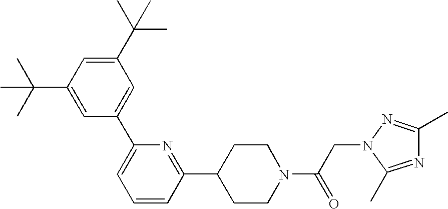

Using the method of Example 2, Step 3 with the product of Example 2, Step 2 and (3,5-dimethyl-1H-1,2,4-triazol-1-yl)acetic acid as starting materials the title compound was obtained.

500 MHz NMR (CDCl₃): δ 7.82 (d, 2H, J=1.8 Hz), 7.71 (t, 1H, J=7.8 Hz), 7.59 (d, 1H, J=7.7 Hz), 7.52 (t, 1H, J=1.7 Hz), 7.09 (d, 1H, J=7.6 Hz), 4.94 (bs, 2H), 4.70 (bd, 1H, J=13.5 Hz), 4.05 (bd, 1H, J=13.5 Hz), 3.34 (dt, 1H, Jₜ=14.2, J_{d}=2.1 Hz), 3.08 (tt, 1H, J=11.4, 3.5 Hz), 2.90 (dt, 1H, J=13.1, J_{d}=1.8 Hz), 2.44 (s, 3H), 2.36 (s, 3H), 2.19 (bd, 1H, J=13.1 Hz), 2.12 (bd, 1H, J=13.1 Hz), 1.94-1.85 (m, 2H), 1.41 (s, 18H). LRMS calc: 487.3 obs: 488.3 (M+H).

### EXAMPLE 5

### Preparation of 3-[2-(4-{6-[3,5-bis(trifluoromethyl)phenyl]-2-pyridinyl}-1-piperidinyl)-2-oxoethyl]-3H-imidazo[4,5-b]pyridine

### Step 1 2-[3,5-bis(trifluoromethyl)phenyl]-6-bromopyridine

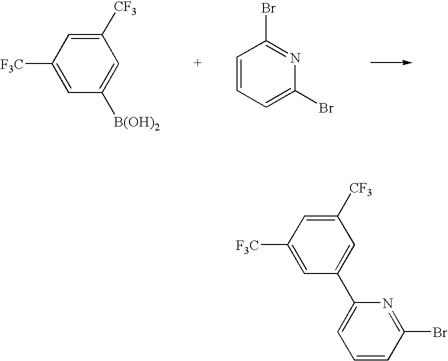

A solution of 2,6-dibromopyridine (490 mg; 2.068 mmol) in THF (4 mL) was treated with 3,5-bis(trifluoromethyl)phenylboronic acid (587 mg; 2.275 mmol). Tetrakis(triphenylphosphine)palladium (0) (120 mg; 0.103 mmol) was added, followed by aq. Na₂CO₃ (12 mL, 2M). The mixture was refluxed for 5 h. The reaction was partitioned between iPrOAc and water. The organic was dried over MgSO₄, filtered and evaporated to a solid. The solid was chromatographed (prep. TLC; 1:1 hex/CH₂Cl₂). The major band was recovered and rechromatographed (prep. TLC; 10:1 hex/MTBE). The major band was again recovered, affording the title compound (233 mg).

500 MHz NMR (CDCl₃): δ 8.49 (s, 2H), 7.98 (s, 1H), 7.82 (d, 1H, J=7.8 Hz), 7.74 (t, 1H, J=7.8 Hz), 7.58 (d, 1H, J=7.8 Hz). LRMS calc: 369.0 obs: 370.0 (M+H).

### Step 2. 2-[3,5-bis(trifluoromethyl)phenyl]-6-(4-piperidinyl)pyridine bis(hydrotrifluoroacetate)

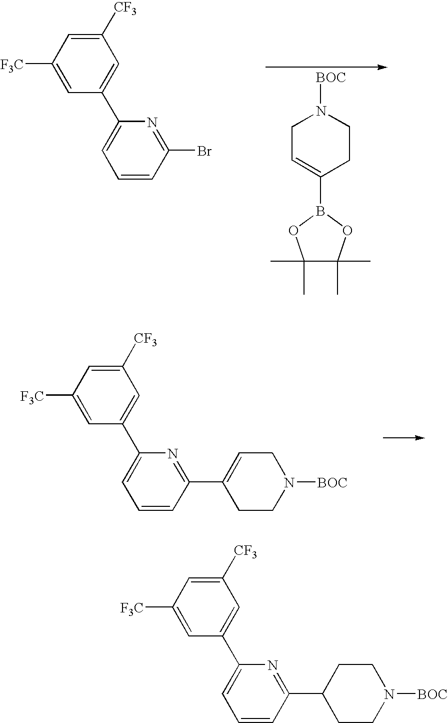

A solution of 2-[3,5-bis(trifluoromethyl)phenyl]-6-bromopyridine (92 mg; 0.248 mmol) in dry DMF (2 mL) was treated with tert-butyl-4-(4,4,5,5-tetramethyl-1,3,2-dioxaborolan-2-yl)-3,6-dihydro-1(2H)-pyridinecarboxylate (73 mg; 0.236 mmol; prepared using the method of Eastman, P. R. Tet. Lett. 2000, 41 (19), 3705). K₂CO₃ (98 mg; 0.708 mmol) was added, followed by PdCl₂(dppf) (10 mg; 0.014 mmol). The mixture was stirred at 80° C. for 16 h. The mixture was partitioned between iPrOAc and aq. NaHCO₃. The organic was dried over MgSO₄, filtered and evaporated to a solid. The solid was chromatographed (prep. TLC; CH₂Cl₂) to give the title compound (24 mg).

500 MHz NMR (CDCl₃): δ 8.51 (s, 2H), 7.92 (s, 1H), 7.81 (t, 1H, J=7.9 Hz), 7.69 (d, 1H, J=7.8 Hz), 7.38 (bd, 1H, J=7.8 Hz), 6.75 (vbs, 1H), 4.20 (dd, 2H, J=5.9, 2.8 Hz), 3.71 (t, 2H, J=5.7 Hz), 2.75 (vbs, 2H), 1.51 (s, 9H). LRMS calc: 448.3 obs: 449.2 (M+H). LRMS calc: 472.2 obs: 473.0 (M+H).

### tert-butyl-4-{6-[3,5-bis trifluoromethyl-phenyl]-2-pyridinyl}-1-piperidinecarboxylate

A solution of tert-butyl-6-[3,5-bis(trifluoromethyl)phenyl]-3′,6′-dihydro-2,4′-bipyridine-1′(2′R)-carboxylate (24 mg; 0.051 mmol) in ethyl acetate (2 mL) was treated with 10% Pd/C hydrogenation catalyst (25 mg). The mixture was shaken under a hydrogen atmosphere (21 psi) for 8 h. The mixture was filtered through Celite and evaporated to give the title compound (24 mg).

500 MHz NMR (CD₃OD): δ 8.64 (s, 2H), 7.99 (bs, 1H), 7.85 (bs, 1H), 7.37 (bs, 1H), 7.22 (bs, 1H), 4.23 (bd, 2H, J=13.0 Hz), 3.05-2.86 (bm, 3H), 1.96 (bd, 2H, J=12.1 Hz), 1.80 (dquart, 2H, J_{q}=12.6, J_{d}=3.9 Hz), 1.49 (s, 9H).

### 2-[3,5-bis(trifluoromethyl)phenyl]-6-(4-piperidinyl)pyridine bis(hydrotrifluoroacetate)

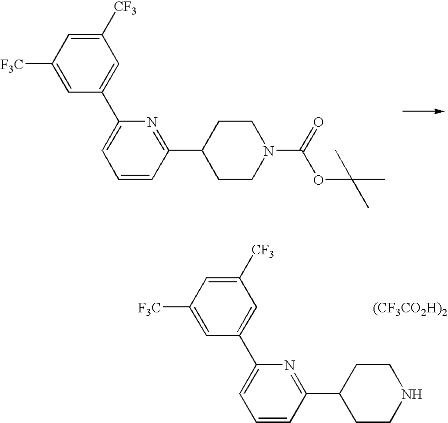

A solution of tert-butyl-4-{6-[3,5-bis(trifluoromethyl)phenyl]-2-pyridinyl}-1-piperidinecarboxylate (24 mg; 0.051 mmol) in dry CH₂Cl₂ (2 mL) was treated with CF₃CO₂H (1 ml). The solution was stirred at ambient temperature for 1 h. The solution was diluted with toluene (5 mL) and evaporated to a residue. The operation was repeated, affording the title compound (28 mg).

500 MHz NMR (CD₃OD): δ 8.64 (s, 2H), 8.01 (s, 1H), 7.95 (t, 1H, J=7.7 Hz), 7.90 (d, 1H, J=7.8 Hz), 7.38 (d, 1H, J=7.7 Hz), 3.57 (dt, 2H, J_{d}=12.0, Jₜ=1.8 Hz), 3.20 (m, 3H), 2.20 (m, 4H).

### Step 3. 3-[2-(4-{6-[3,5-bis(trifluoromethyl)phenyl]-2-pyridinyl}-1-piperidinyl)-2-oxoethyl]-3H-imidazo[4,5-b]pyridine

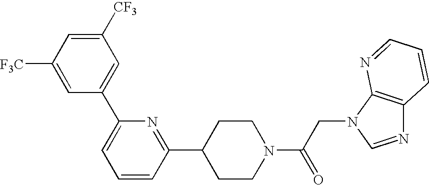

Using the method of Example 2, Step 3 with the products of Example 5, Step 2 and Example 1 Step 3, (3H-imidazo[4,5-b]pyridine-3-yl)acetic acid, as starting materials the title compound was obtained.

600 MHz NMR (CDCl₃): δ 8.48 (s, 2H), 8.39 (dd, 1H, J=4.7, 1.0 Hz), 8.25 (s, 1H), 8.11 (d, 1H, J=7.9 Hz), 7.93 (s, 1H), 7.80 (t, 1H, J=7.9 Hz), 7.69 (d, 1H, J=7.8 Hz), 7.26 (dd, 1H, J=8.0, 4.7 Hz), 7.22 (d, 1H, J=7.7 Hz), 5.26 (½AB, 1H, J=17.0 Hz), 5.22 (½AB, 1H, J=16.9 Hz), 4.76 (bd, 1H, J=13.4 Hz), 4.20 (bd, 1H, J=13.4 Hz), 3.41 (dt, 1H, Jₜ=13.2, J_{d}=2.5 Hz), 3.11 (ft, 1H, J=12.0, 3.5 Hz), 2.88 (dt, 1H, Jₜ=13.0, 2.6 Hz), 2.19 (bd, 1H, J=13.3 Hz), 2.09 (bd, 1H, J=13.2 Hz), 1.96 (dquart, 1H, J_{q}=12.6, J_{d}=4.0 Hz), 1.87 (dquart, 1H, J_{q}=12.7, J_{d}=3.9 Hz). LRMS calc: 533.2 obs: 534.1 (M+H).

### EXAMPLE 6

### Preparation of 3-(2-{4-[6-(3,5-di-tert-butyl-4-methoxyphenyl)-2-pyridinyl]-1-piperidinyl}-2-oxoethyl)-3H-imidazo[4,5-b]pyridine

### Step 1. 2-bromo-6-(3,5-di-tert-butyl-4-methoxyphenyl)pyridine

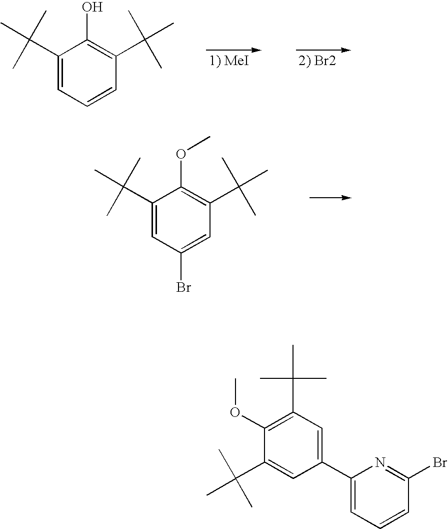

### 1,3-di-tert-butyl-2-methoxybenzene

A solution of 2,6-di-tert-butylphenol (2.014 g; 9.761 mmol) in dry DMF (30 mL) was treated with methyl iodide (3.64 mL; 58.57 mmol) followed by Cs₂CO₃ (3.66 g; 11.225 mmol). The mixture was stirred at ambient temperature for 16 h. The mixture was partitioned between iPrOAc and water. The organic was washed twice more with water, dried over MgSO₄, filtered and evaporated to give the title compound (2.034 g).

500 MHz NMR (CDCl₃): δ 7.26 (d, 2H, J=7.7 Hz), 7.02 (t, 1H, J=7.9 Hz), 3.75 (s, 3H), 1.44 (s, 18H).

### 5-bromo-1,3-di-tert-butyl-2-methoxybenzene

A −10° C. solution of 1,3-di-tert-butyl-2-methoxybenzene (2.034 g; 9.231 mmol) in propionic acid (20 mL) was treated with a solution of bromine (14.8 mL; 0.78M in propionic acid; 11.56 mmol). The cold bath was removed and the solution stirred at ambient temperature for 16 h. The solution was partitioned between iPrOAc and water. The organic was washed once more with water, then twice with aq. NaHCO₃. The organic was dried over MgSO₄, filtered and evaporated to a solid. The solid was chromatographed over silica get (0% to 40% CH₂Cl₂/hex). The most mobile product was collected giving the title compound (2.142 g).

500 MHz NMR (CDCl₃): δ 7.38 (s, 2H), 3.73 (s, 3H), 1.46 (s, 18H).

### 2-bromo-6-(3,5-di-tert-butyl-4-methoxyphenyl)pyridine

A −78° C. solution of 5-bromo-1,3-di-tert-butyl-2-methoxybenzene (2.142 g; 7.158 mmol) in dry THF (15 mL) was treated with n-butyllithium (7.00 mL, 1.13M in hex, 7.88 mmol). The solution was stirred at −78° C. for 15 min, then treated with trimethylborate (976 μL; 8.59 mmol). The reaction was allowed to warm to ambient temperature and evaporated to a thick oil. The oil was dissolved in DME (15 mL). 2,6-dibromopyridine (1.542 g; 6.507 mmol) was added, followed by tetrakis(triphenylphosphine)palladium (0) (376 mg; 0.323 mmol). Aq. Na₂CO₃ (40 mL, 2M) was added and the mixture refluxed for 16 h. The mixture was partitioned between iPrOAc and water. The organic was dried over MgSO₄, filtered and evaporated to a residue. The crude product was chromatographed over silica gel (0% to 40% MTBE/hex; linear gradient). The major product fractions were recovered and rechromatographed (prep. TLC; 1:1 hex/CH₂Cl₂). The most polar mobile band was recovered to give the title compound (1.250 mg).

500 MHz NMR (CDCl₃): δ 7.86 (s, 2H), 7.63 (dd, 1H, J=7.8, 0.7 Hz), 7.58 (t, 1H, J=7.8 Hz), 7.38 (dd, 1H, J=7.8, 0.7 Hz), 3.74 (s, 3H), 1.51 (s, 18H). LRMS calc: 375.1 obs: 376.1 (M+H).

### Step 2. 2-(3,5-di-tert-butyl-4-methoxyphenyl)-6-(4-piperidinyl)pyridine bis(hydrotrifluoroacetate)

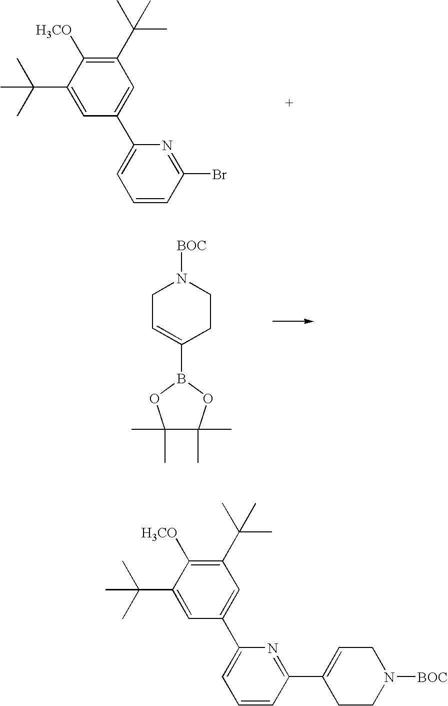

### tert-butyl-6-(3,5-di-tert-butyl-4-methoxyphenyl)-3′,6′-dihydro-2,4′-bipyridine-1′(2′H-carboxylate

A solution of 2-bromo-6-(3,5-di-tert-butyl-4-methoxyphenyl)pyridine (795 mg; 2.113 mmol) in DME (6 mL) was treated with tert-butyl-4-(4,4,5,5-tetramethyl-1,3,2-dioxaborolan-2-yl)-3,6-dihydro-1(2H)-pyridinecarboxylate (980 mg; 3.171 mmol; prepared using the method of Eastman, P. R. Tet. Lett. 2000, 41 (19), 3705). Tetrakis(triphenylphosphine)palladium (0) (244 mg; 0.211 mmol) was added, followed by aq. Na₂CO₃ (15 mL, 2M). The mixture was refluxed for 5 h. The mixture was partitioned between iPrOAc and water. The organic was dried over MgSO₄, filtered and evaporated to a solid. The solid was chromatographed (prep. TLC; CH₂Cl₂). The major product band was recovered and rechromatographed (prep. TLC; 4:1 hex/MTBE). The most polar fluorescent band was recovered to give the title compound (56 mg).

500 MHz NMR (CDCl₃): δ 7.96 (s, 2H), 7.71 (t, 1H, J=7.8 Hz), 7.57 (d, 1H, J=7.8 Hz), 7.30 (t, 1H, J=7.7 Hz), 6.76 (vbs, 1H), 4.20 (bd, 2H, J=2.5 Hz), 3.75 (s, 3H), 3.71 (vbs, 2H), 2.78 (vbd, 2H, J=1.1 Hz), 1.53 (s, 9H), 1.52 (s, 18H). LRMS calc: 478.3 obs: 479.3 (M+H).

### 2-(3,5-di-tert-butyl-4-methoxyphenyl)-6-(4-piperidinyl)pyridine bis(hydrotrifluoroacetate)

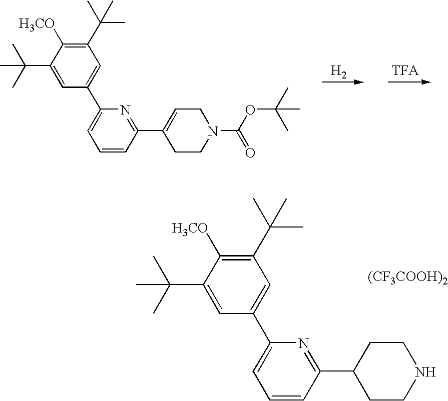

A solution of tert-butyl-6-(3,5-di-tert-butyl-4-methoxyphenyl)-3′,6′-dihydro-2,4′-bipyridine-1′(2′H)-carboxylate (56 mg; 0.117 mmol) in ethanol (4 mL) was treated with 10% Pd/C hydrogenation catalyst (60 mg). The mixture was shaken under a hydrogen atmosphere (50 psi) for 1.5 h. The mixture was filtered through Celite and evaporated to give the title compound (57 mg).

500 MHz NMR (CDCl₃): δ 7.92 (s, 2H), 7.65 (vbs, 1H), 7.57 (vbs, 1H), 7.06 (vbs, 1H), 4.24 (vbs, 2H), 3.77 (s, 3H), 2.97 (vbs, 3H), 2.05 (vbs, 2H), 1.81 (vbs, 2H), 1.54 (s, 9H), 1.53 (s, 18H). LRMS calc: 480.4 obs: 481.4 (M+H).

A solution of tert-butyl-4-[6-(3,5-di-tert-butyl-4-methoxyphenyl)-2-pyridinyl]-1-piperidinecarboxylate (57 mg; 0.117 mmol) in dry CH₂Cl₂ (3 mL) was treated with CF₃CO₂H (1 ml). The solution was stirred at ambient temperature for 2 h. The solution was diluted with toluene (5 mL) and evaporated to a residue. The operation was repeated, affording the title compound (59 mg).

500 MHz NMR (CD3OD): δ 8.49 (t, 1H, J=8.0 Hz), 8.08 (d, 1H, J=8.0 Hz), 7.87 (d, 1H, J=7.8 Hz), 7.77 (t, 1H, J=1.7 Hz), 7.74 (d, 2H, J=1.8 Hz), 3.62-3.54 (m, 3H), 3.19 (dt, 2H, Jₜ=12.8, J_{d}=2.3 Hz), 2.31 (bd, 2H, J=14.2 Hz), 2.17 (dquart, 2H, J_{q}=13.5, J_{d}=3.5 Hz), 1.41 (s, 18H). LRMS calc: 380.3 obs: 381.4 (M+H).

### Step 3. 3-(2-{4-[6-(3,5-di-tert-butyl-4-methoxyphenyl)-2-pyridinyl]-1-piperidinyl}-2-oxoethyl)-3H-imidazo[4,5-b]pyridine

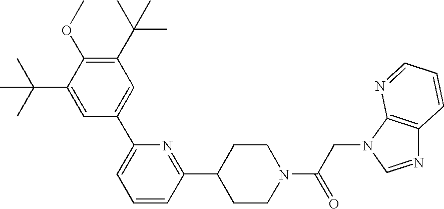

Using the method of Example 2, Step 3 with the products of Example 6, Step 2 and Example 1 Step 3, (3H-imidazo[4,5-b]pyridine-3-yl)acetic acid, as starting materials the title compound was obtained.

500 MHz NMR (CDCl₃): δ 8.40 (d, 1H, J=3.9 Hz), 8.26 (bs, 1H), 8.13 (bd, 1H, J=7.7 Hz), 7.90 (s, 2H), 7.70 (t, 1H, J=7.8 Hz), 7.54 (d, 1H, J=7.7 Hz), 7.27 (dd, 1H, J=7.8, 4.8 Hz), 7.07 (d, 1H, J=7.7 Hz), 5.26 (½AB, 1H, J=16.2 Hz), 5.22 (½AB, 1H, J=16.4 Hz), 4.71 (bd, 1H, J=13.1 Hz), 4.19 (bd, 1H, J=13.5 Hz), 3.75 (s, 3H), 3.43 (dt, 1H, Jₜ=12.4, J_{d}=2.5 Hz), 3.09 (tt, 1H, J=11.5, 3.7 Hz), 2.95 (dt, 1H, Jₜ=13.1, J_{d}=2.0 Hz), 2.22 (bd, 1H, J=13.1 Hz), 2.13 (bd, 1H, J=13.0 Hz), 1.96 (dquart, 1H, J_{q}=12.8, J_{d}=3.6 Hz), 1.90 (dquart, 1H, J_{q}=13.2, J_{d}=4.0 Hz), 1.52 (s, 18H). LRMS calc: 539.3 obs: 540.4 (M+H).

### EXAMPLE 7

### Preparation of 2-(3,5-di-tert-butyl-4-methoxyphenyl)-6-{1-[(3,5-dimethyl-1H-pyrazol-1-yl)acetyl]-4-piperidinyl}pyridine

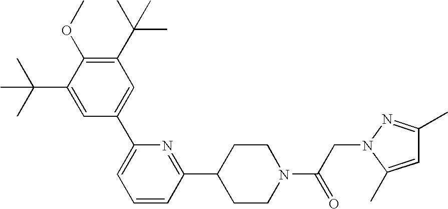

Using the method of Example 2, Step 3 with the product of Example 6, Step 2 and (3,5-dimethyl-1H-pyrazol-1-yl)acetic acid as starting materials the title compound was obtained.

500 MHz NMR (CDCl₃): δ 7.90 (s, 2H), 7.69 (t, 1H, J=7.8 Hz), 7.54 (d, 1H, J=7.7 Hz), 7.05 (d, 1H, J=7.5 Hz), 5.88 (bs, 1H), 4.95 (½AB, 1H, J=16.0 Hz), 4.91 (½AB, 1H, J=16.1 Hz), 4.71 (bd, 1H, J=13.1 Hz), 4.12 (bd, 1H, J=13.3 Hz), 3.75 (s, 3H), 3.28 (dt, 1H, Jₜ=12.3, J_{d}=2.3 Hz), 3.04 (tt, 1H, J=11.7, 3.6 Hz), 2.86 (dt, 1H, Jₜ=12.8, J_{d}=2.5 Hz), 2.27 (s, 3H), 2.24 (s, 3H), 2.11 (bt, 2H, J=15.6 Hz), 1.92-1.80 (m, 2H), 1.52 (s, 18H). LRMS calc: 516.4 obs: 517.4 (M+H).

### EXAMPLE 8

### Preparation of 2-(3,5-di-tert-butyl-4-methoxyphenyl)-6-{1-[(2,4-dimethyl-1H-imidazol-1-yl)acetyl]-4-piperidinyl}pyridine

### Step 1. 2,4-dimethyl-1H-imidazol-1-ylacetic acid

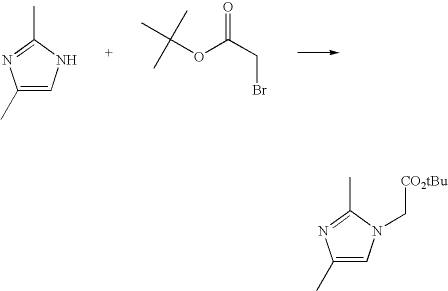

A solution of 2,4-dimethylimidazole (8-40 g; 87.38 mmol) in dry DMF (250 mL) was treated with potassium tert-butoxide (9.806 g; 87.38 mmol). The mixture was stirred at ambient temperature until homogenous. tert-Butyl bromoacetate (15.29 mL; 104.9 mmol) was added dropwise. The solution was stirred for 15 min, then diluted with isopropyl acetate (400 mL) and washed with pH7 phosphate buffer (3×250 mL). The organic was dried over MgSO₄, filtered and evaporated to an oil. The crude product (consisting of the two title compounds) was chromatographed over silica gel (0% to 10% MeOH/CH₂Cl₂; linear gradient). All fractions containing the two products were combined. The residue was rechromatographed on Chiralcel OD stationary phase (Daicel Chemical Industries Ltd., Chiralcel Technologies Inc.; 10% ethanol/heptane; λ=220 nM). The more mobile 2,5 isomer and less mobile 2,4 isomer were obtained.

500 MHz ¹H NMR (CDCl₃): (2,4 isomer) δ 6.49 (s, 1H), 4.39 (s, 2H), 2.24 (s, 3H), 2.17 (s, 3H), 1.42 (s, 9H); (2,5 isomer) δ 6.67 (s, 1H), 4.41 (s, 2H), 2.35 (s, 3H), 2.15 (s, 3H), 1.48 (s, 9H).

A solution of 2,4-dimethyl-1H-imidazol-1-ylacetic acid tert-butyl ester (1.07 g; 5.09 mmol) in CH₂Cl₂ (15 mL) was treated with trifluoroacetic acid (30 mL) at ambient temperature. The solution was stirred for 1 h. The volatiles were removed and the residue flushed with toluene (2×15 mL) affording the title compound as an oil (1.32 g).

500 MHz ¹H NMR (CD₃OD): δ 7.20 (s, 1H), 5.03 (s, 2H), 2.59 (s, 3H), 2.32 (s, 3H).

### Step 2. 2-(3,5-di-tert-butyl-4-methoxyphenyl)-6-{1-[(2,4-dimethyl-1H-imidazol-1-yl)acetyl]-4-piperidinyl}pyridine

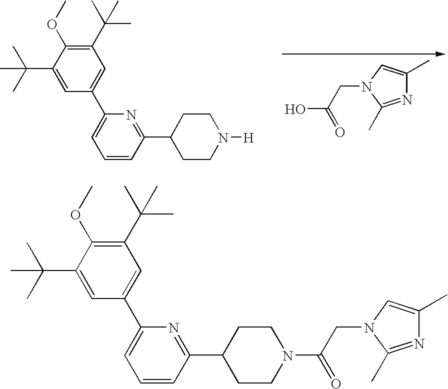

Using the method of Example 2, Step 3 with the product of Example 6 Step 2 and (2,4-dimethyl-1H-imidazol-1-yl)acetic acid, from Example 8, Step 1, as starting materials the title compound was obtained.

500 MHz NMR (CDCl₃): δ 7.90 (s, 2H), 7.69 (t, 1H, J=7.8 Hz), 7.54 (d, 1H, J=7.8 Hz), 7.06 (d, 1H, J=7.7 Hz), 6.57 (bs, 1H), 4.72-4.66 (bd overlapping AB, 3H total), 3.94 (bd, 1H, J=13.5 Hz), 3.75 (s, 3H), 3.33 (bt, 1H, J=11.7 Hz), 3.07 (tt, 1H, J=11.4, 3.7 Hz), 2.92 (bt, 1H, J=12.6 Hz), 2.38-2.34 (s overlapping bd, 4H total), 2.22 (s, 3H), 2.11 (bd, 1H, J=11.2 Hz), 1.90 (vbquart, 2H, J=12.3 Hz), 1.51 (s, 18H). LRMS calc: 516.4 obs: 517.4 (M+H).

### EXAMPLE 9

### Preparation of 3-[2-(4-{4-[3,5-bis(trifluoromethyl)phenyl]-2-pyrimidinyl}-1-piperidinyl)-2-oxoethyl]-3H-imidazo[4,5-b]pyridine

### Step 1. 4-[3,5-bis(trifluoromethyl)phenyl]-2-chloropyrimidine

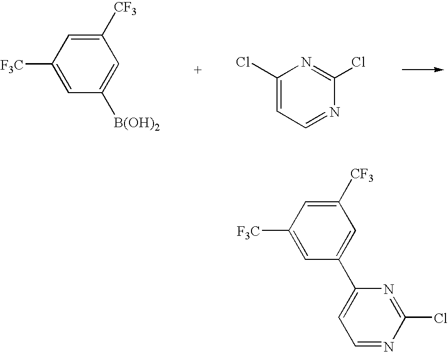

A solution of 2,4-dichloropyrimidine (186 mg; 1.248 mmol) in THF (2 mL) was treated with 3,5-bis(trifluoromethyl)phenylboronic acid (354 mg; 1.373 mmol). Tetrakis(triphenylphosphine)palladium (0) (72 mg; 0.062 mmol) was added, followed by aq. Na₂CO₃ (6 mL, 2M). The mixture was refluxed for 5 h. The reaction was partitioned between iPrOAc and water. The organic was dried over MgSO₄, filtered and evaporated to a solid. The solid was chromatographed (prep. TLC; CH₂Cl₂). The major, middle band was recovered, affording the title compound (280 mg).

500 MHz NMR (CDCl₃): δ 8.80 (d, 1H, J=5.9 Hz), 8.58 (s, 2H), 8.06 (s, 1H), 7.79 (d, 1H, J=5.8 Hz). LRMS calc: 326.0 obs: 327.0 (M+H).

### Step 2. 4-[3,5-bis(trifluoromethyl)phenyl]-2-(4-piperidinyl)pyrimidine bis(hydrotrifluoroacetate)

### tert-butyl-4-{-4-[3,5-bis(trifluoromethyl)phenyl]-2-pyrimidinyl}-3,6-dihydro-1-(2H)-pyridinecarboxylate

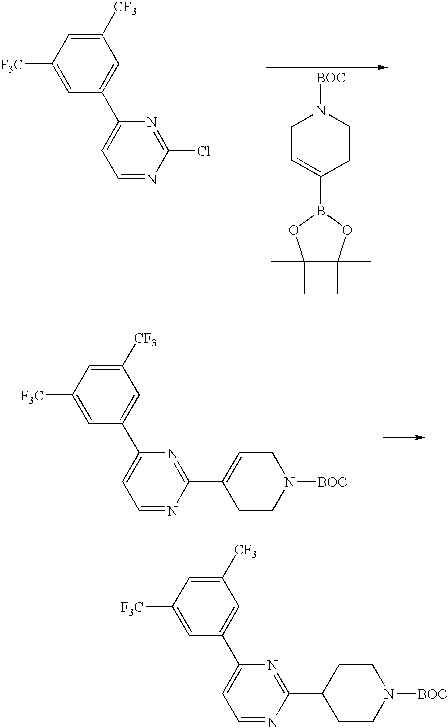

A solution of 4-[3,5-bis(trifluoromethyl)phenyl]-2-chloropyrimidine (111 mg; 0.340 mmol) in dry DME (2 mL) was treated with tert-butyl-4-(4,4,5,5-tetramethyl-1,3,2-dioxaborolan-2-yl)-3,6-dihydro-1(2H)-pyridinecarboxylate (105 mg; 0.340 mmol; prepared using the method of Eastman, P. R. Tet. Lett. 2000, 41 (19), 3705). Tetrakis(triphenylphosphine)palladium (0) (39 mg; 0.034 mmol) was added, followed by aq. Na₂CO₃ (6 mL, 2M). The mixture was refluxed for 16 h. The mixture was partitioned between iPrOAc and pH7 phosphate buffer. The organic was dried over MgSO₄, filtered and evaporated to a solid. The solid was chromatographed (prep. TLC; 10:1 CH₂Cl₂/EtOAc). The major, middle band was recovered to give the title compound (50 mg).

500 MHz NMR (CDCl₃): δ 8.87 (d, 1H, J=5.0 Hz), 8.60 (s, 2H), 8.04 (s, 1H), 7.63 (d, 1H, J=5.1 Hz), 7.39 (vbs, 1H), 4.27 (bd, 2H, J=2.3 Hz), 3.72 (bt, 2H, J=5.3 Hz), 2.85 (bs, 2H), 1.53 (s, 9H). LRMS calc: 473.2 obs: 474.1 (M+H).

### tert-butyl-4-{4-[3,5-bis(trifluoromethyl)phenyl]-2-pyrimidinyl}-1-piperidinecarboxylate

A solution of tert-butyl-4-{4-[3,5-bis(trifluoromethyl)phenyl]-2-pyrimidinyl}-3,6-dihydro-1-(2H)-pyridinecarboxylate (50 mg; 0.105 mmol) in ethyl acetate (3 mL) was treated with 10% Pd/C hydrogenation catalyst (105 mg). The mixture was shaken under a hydrogen atmosphere (50 psi) for 30 h. The mixture was filtered through Celite and evaporated to give the title compound (30 mg).

500 MHz NMR (CDCl₃): δ 8.86 (d, 1H, J=5.3 Hz), 8.58 (s, 2H), 8.04 (s, 1H), 7.66 (d, 1H, J=5.2 Hz), 4.26 (vbs, 2H), 3.17 (tt, 1H, J=11.7, 3.4 Hz), 2.95 (bs, 2H), 2.09 (bt, 2H, J=11.2 Hz), 1.93 (dquart, 2H, J_{q}=12.6, J_{d}=4.0 Hz), 1.52 (s, 9H). LRMS calc: 475.2 obs: 476.0 (M+H).

### 4-[3,5-bis(trifluoromethyl)phenyl]-2-(4-piperidinyl)pyrimidine bis(hydrotrifluoroacetate)

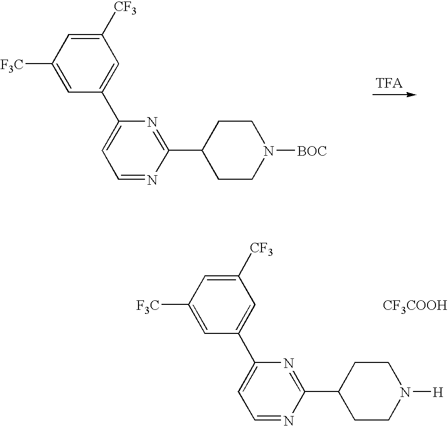

A solution of tert-butyl-4-{4-[3,5-bis(trifluoromethyl)phenyl]-2-pyrimidinyl}-1-piperidinecarboxylate (30 mg; 0.063 mmol) in dry CH₂Cl₂ (2 mL) was treated with CF₃CO₂H (1 ml). The solution was stirred at ambient temperature for 1 h. The solution was diluted with toluene (5 mL) and evaporated to a residue. The operation was repeated, affording the title compound (30 mg). 500 MHz NMR (CD₃OD): δ 8.92 (d, 1H, J=5.0 Hz), 8.80 (s, 2H), 8.18 (s, 1H), 8.05 (d, 1H, J=5.0 Hz), 3.57 (dt, 2H, J_{d}=11.5, Jₜ=2.8 Hz), 3.38 (tt, 1H, J=11.3, 3.1 Hz), 3.23 (dt, 2H, Jₜ=1.6, J_{d}=2.5 Hz), 2.37 (bdd, 2H, J=11.6, 3.0 Hz), 2.21 (dquart, 2H, J_{q}=11.9, J_{d}=3.3 Hz). LRMS calc: 375.1 obs: 376.0 (M+H).

### Step 3. 3-[2-(4-{4-[3,5-bis(trifluoromethyl)phenyl]-2-pyrimidinyl}-1-piperidinyl)-2-oxoethyl]-3H-imidazo[4,5-b]pyridine

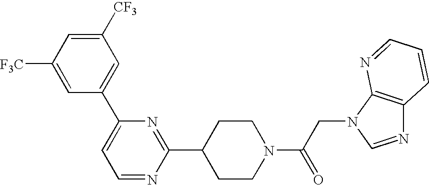

Using the method of Example 2, Step 3 with the products of Example 9, Step 2 and Example 1 Step 3, (3H-imidazo[4,5-b]pyridine-3-yl)acetic acid, as starting materials the title compound was obtained.

500 MHz NMR (CDCl₃): δ 8.87 (d, 1H, J=5.2 Hz), 8.57 (s, 2H), 8.41 (dd, 1H, J=4.8, 1.4 Hz), 8.26 (s, 1H), 8.12 (dd, 1H, J=8.0, 1.4 Hz), 8.05 (s, 1H), 7.68 (d, 1H, J=5.2 Hz), 7.27 (dd, 1H, J=8.0, 4.8 Hz), 5.27 (½AB, 1H, J=16.2 Hz), 5.23 (½AB, 1H, J=16.4 Hz), 4.72 (bd, 1H, J=13.5 Hz), 4.20 (bd, 1H, J=13.5 Hz), 3.45 (dt, 1H, J=13.5, J_{d}=2.7 Hz), 3.32 (tt, 1H, J=11.4, 3.6 Hz), 2.97 (dt, 1H, J=13.0, 2.9 Hz), 2.29 (bd, 1H, J=12.6 Hz), 2.23 (bd, 1H, J=12.2 Hz), 2.09 (dquart, 1H, J_{q}=12.8, J_{d}=3.9 Hz), 1.97 (dquart, 1H, J_{q}=13.0, J_{d}=4.1 Hz). LRMS calc: 534.2 obs: 535.1 (M+H).

### EXAMPLE 10

### Preparation of 3-(2-{4-[2-(3,5-ditert-butyl-4-methoxyphenyl)pyrimidin-4-yl]piperidin-1-yl}-2-oxoethyl)-3H-imidazo[4,5-b]pyridine

### Step 1. tert-butyl 4-[3-(trimethylsilyl)prop-2-ynoyl]piperidine-1-carboxylate

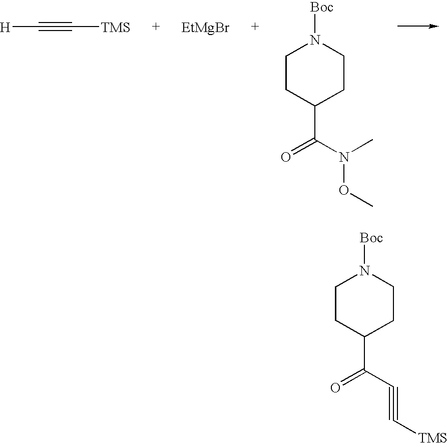

Trimethylsilylacetylene (0.565 mL, 4 mmole) was added slowly to a solution of EtMgBr (4 mL, 4 mmole) in THF at 0° C., the mixture was allowed to stir for 0.5 hr at this temperature. The mixture was added to tert-butyl 4-{[methoxy(methyl)amino]carbonyl}piperidine-1-carboxylate (1.08 g, 4 mmole) in THF (4 mL) dropwise at 0° C. After stirring at this temperature for 30 min, the reaction was slowly warmed to room temperature, quenched with sat. ammonium chloride, extracted with ethyl acetate (3×5 mL), washed with brine, dried over sodium sulfate, reduced i. vac. Purification by column chromatography gave a off white oil as product (0.96 g, 77%).

### Step 2. 3,5-di-tert-butyl-4-methoxybenzenecarboximidamide

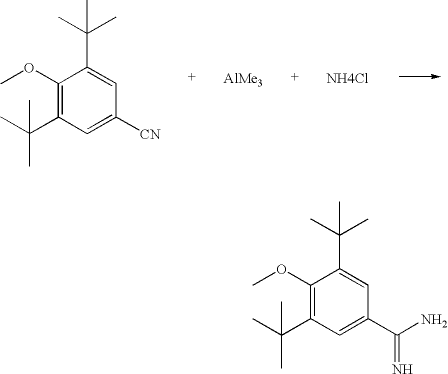

To a solution of ammonium chloride (144 mg, 2.7 mmole) in p-xylene (2 mL) was added trimethyl aluminum dropwise at 0° C. The mixture was stirred for 0.5 hr at this temperature and allowed to warm to room temperature. 3,5-di-tert-butyl-4-methoxybenzonitrile (246 mg, 1 mmole) was then added. The reaction was heated at refluxed for 24 hrs before being allowed to cool. The mixture was poured onto a slurry of silica gel (2 g) and chloroform (2 mL). The silica gel was filtered and washed with MeOH/CHCl₃ (1:1, 20 mL), the filtrate was collected, and concentrated to give the titled compound.

### Step 3. tert-butyl 4-[2-(3,5-di-tert-butyl-4-methoxyphenyl)pyrimidin-4-yl]piperidine-1-carboxylate

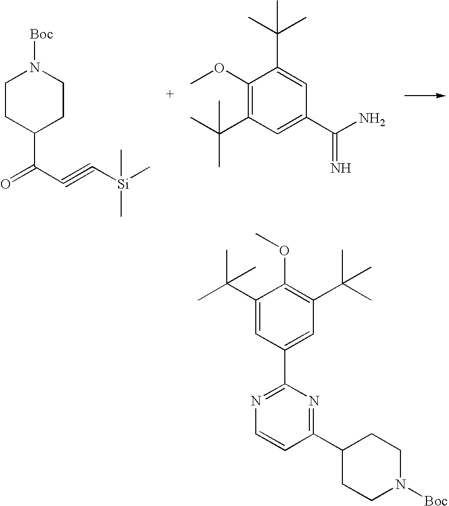

The amidine prepared in Example 10 Step 2 (314 mg, 1.2 mmole) was added to a suspension of the alkyne prepared in Example 10 Step 1 (309 mg, 1 mmole) and sodium carbonate (270 mg, 2.5 mmole) in acetonitrile (5 mL). The mixture was stirred for 5 hrs at 120° C. in a microwave reactor. The mixture was filtered, and diluted with water (1.5 mL), HPLC purification gave off white oil as product. (167 mg, 35%).

### Step 4. 3-(2-{4-[2-(3,5-di-tert-butyl-4-methoxyphenyl)pyrimidin-4-yl]piperidin-1-yl}-2-oxoethyl)-3H-imidazo[4,5-b]pyridine

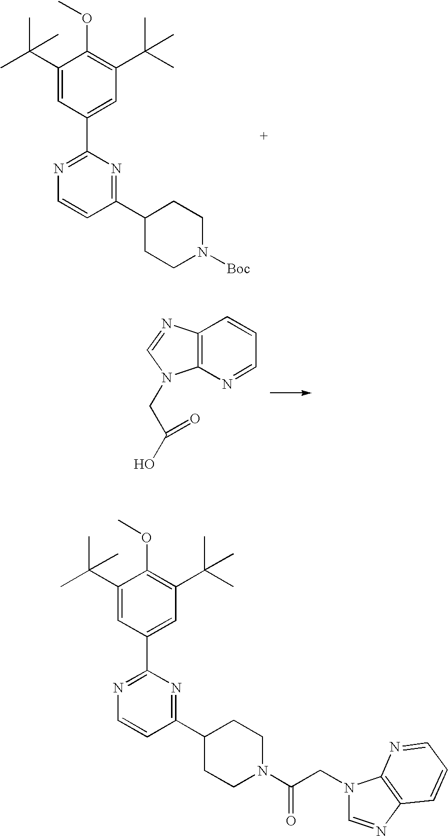

The BOC piperidine prepared in Example 10 Step 3 (24 mg, 005 mmol) was dissolved in trifluoroacetic acid (2 ml) and stirred for 10 minutes. The volatiles were removed i. vac. The residue was dissolved in ethyl acetate, washed with 4:1 water/saturated sodium bicarbonate (40 ml) and extracted (3 times) with ethyl acetate. The combined organic fraction was washed with brine, dried over sodium sulfate, filtered and reduced i. vac.

The unpurified amine was combined with 1-hydroxybenzotriazole (9.4 mg, 0.07 mmol) and the acid prepared according to the procedure of Example 1 Step 3 10.6 mg, 0.06 mmol) and dissolved in dimethylformamide (0.5 ml). Diisopropyl ethyl amine (30 mg, 0.23 mmol) and 1-(3-dimethylaminopropyl)-3-ethylcarbodiimide hydrochloride (13 mg, 0.07 mmol) were added and the solution allowed to stir overnight. The reaction mixture was diluted in 2:1 acetonitrile:water (6 ml) and purified by RP-18 HPLC (acetonitrile:H₂O 15 minute gradient 10 to 100%:0.1% trifluoroacetic acid) to give the titled compound.

500 MHz NMR (CD₃ OD) δ 9.30 (s, br, 1H), 8.73 (d, 1H, J=5.0 Hz), 8.62 (d, 1H, J=4.0 Hz), 8.38 (s, 2H), 8.31 (d, 1H, J=8.0 Hz), 7.65 (m, 1H), 7.31 (d, 1H, J=5.5 Hz), 5.68 (d, 1H, J=18.1 Hz), 5.59 (d, 1H, J=18.1 Hz), 4.61 (d, 1H, J=12.5 Hz), 4.27 (d, 1H, J=14 Hz), 3.75 (s, 3H), 3.53 (d, 1H, 13 Hz), 3.18 (t, 1H, 3.1 Hz), 3.02 (t, 1H, 12.0 Hz), 2.26 (d, 1H, J=8 Hz), 2.09 (m, 2H), 1.90 (d, 1H, J=8.2 Hz), 1.49 (s, 18H)

MS: m/z=541.5 (M+H).

### EXAMPLE 11

### Preparation of [1-(2-{4-[2-(3,5-di-tert-butyl-4-methoxyphenyl)pyrimidin-4-yl]piperidin-1-yl}-2-oxoethyl)-5-methyl-1H-pyrazol-3-yl]acetic acid

### Step 1 methyl 3,5-dioxohexanoate

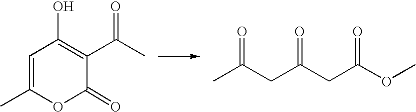

A solution of dehydroacetic acid (20.60 g; 168 mmol) in methanol (400 mL) was treated with a solution of magnesium methoxide (6 wt % in methanol; 350 mL; 184 mmol) at ambient temperature. The reaction was refluxed for 5 h. The solvent was removed and the residue added to aq. HCl (1 L; 1N). The aqueous was extracted (EtOAc, 2×500 mL). The organic was dried over MgSO₄, filtered and evaporated to give the title compound (17.0 g).

### Step 2 (5-methyl-1H-pyrazol-3-yl)acetic acid methyl ester

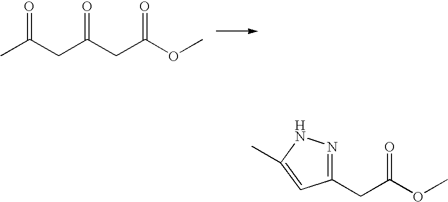

A solution of the product of Example 11, Step 1 (6.0 g; 38 mmol) in ethanol (40 mL) was treated dropwise with hydrazine monohydrate (2.21 mL; 46 mmol) at ambient temperature. The reaction was refluxed for 3 h. The solvent was removed and the residue purified by chromatography on silica gel (CH₂Cl₂/acetone/acetic acid; 30:10:1) to give the title compound (2.3 g).

### Step 3 benzyl methyl 2,2′-(5-methyl-1H-pyrazole-1,3-diyl)diacetate

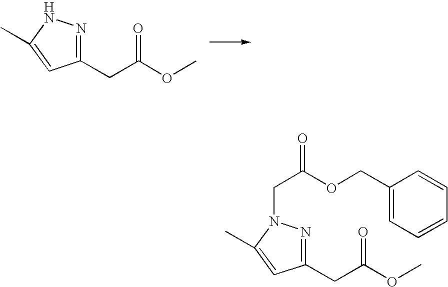

A solution of the product of Example 11, Step 2 (462 mg; 3.0 mmol) in dry DMF (8 mL) was treated with potassium carbonate (415 mg; 3.0 mmol) at ambient temperature. The mixture was warmed to 50° C. Benzyl 2-bromoacetate (687 mg; 3.0 mmol) was added dropwise. The reaction mixture was stirred for 4 h then cooled to ambient and stirred for 16 h. The mixture was diluted with water and extracted (EtOAc). The organic was dried over MgSO₄, filtered and evaporated to a residue. Silica gel chromatography (EtOAc/hexanes; 1:2) gave a mixture of two isomers. The mixture was purified by chromatography on Chiralcel OJ stationary phase (60% ethanol/heptane; λ=220 nM) to give the title compound (194 mg).

### Step 4 [3-(2-methoxy-2-oxoethyl)-5-methyl-1H-pyrazol-1-yl]acetic acid

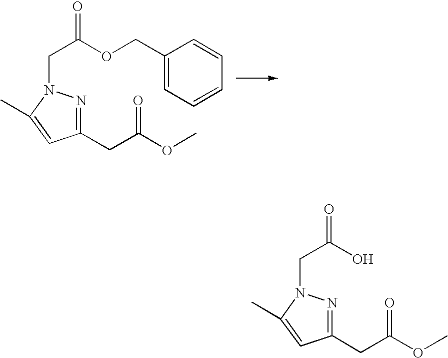

A solution of the product of Example 11, Step 3 (194 mg; 0.64 mmol) in methanol (50 mL) was combined with 10% palladium on carbon hydrogenation catalyst (60 mg). The mixture was shaken under a hydrogen atmosphere (1 atm) for 2 h. The mixture was filtered through Celite and the filtrate concentrated to give the title compound (118 mg).

### Step 5. [1-(2-{4-[2-(3,5-di-tert-butyl-4-methoxyphenyl)pyrimidin-4-yl]piperidin-1-yl}-2-oxoethyl)-5-methyl-1H-pyrazol-3-yl]acetic acid

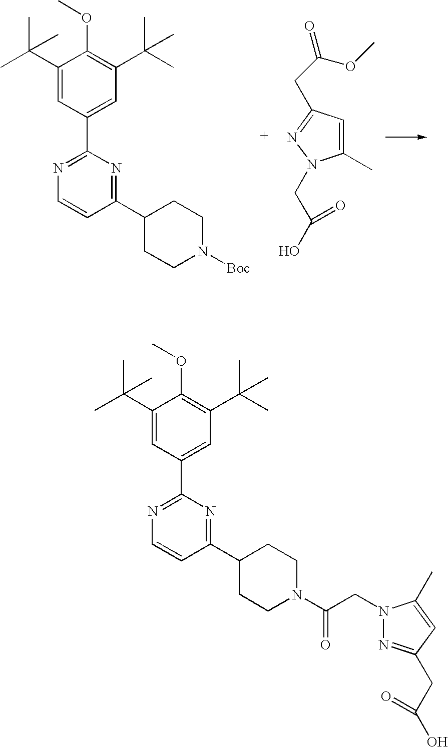

The Boc-protected amine prepared according to the procedure of Example 10 Step 3 (29 mg, 0.06 mmol) was dissolved in trifluoroacetic acid (2 ml) and stirred for 10 minutes. The volatiles were removed i. vac. The residue was dissolved in ethyl acetate, washed with 4:1 water/saturated sodium bicarbonate (40 ml) and extracted (3 times) with ethyl acetate. The combined organic fraction was washed with brine, dried over sodium sulfate, filtered and reduced i. vac.

The unpurified amine (24.2 mg, 0.06) was combined with 1-hydroxybenzotriazole (9.8 mg, 0.07 mmol) and the acid prepared according to the procedure of Example 11 Step 4 (12.7 mg, 0.06 mmol) and dissolved in dimethylformamide (0.5 mL). Diisopropyl ethyl amine (29.5 mg, 0.23 mmol) and 1-(3-dimethylaminopropyl)-3-ethylcarbodiimide hydrochloride (14.0 mg, 0.07 mmol) were added and the solution allowed to stir overnight. The reaction mixture was diluted with water (6 mL), extracted with diethyl ether, the organic layer was washed with water (2×1 mL), concentrated under vacuo. The residue was redissolved in a mixture of MeOH (0.5 mL) and NaOH (1N aq., 0.07 mL), stir 6 hrs at room temperature, MeoH was removed under vacuo. The mixture was diluted in 2:1 acetonitrile:water (6.2 ml) and purified by RP-18 HPLC (acetonitrile:H₂O 15 minute gradient 10 to 100%:0.1% trifluoroacetic acid) to give the titled compound.

500 MHz NMR (CD₃OD) δ 8.73 (d, 1H, J=5.5 Hz), 8.38 (s, 2H), 8.38 (s, 2H), 7.29 (d, 1H, J=5.5 Hz), 6.10 (m, 1H), 5.09 (br, 2H), 4.61 (d, 1H, J=12.5 Hz), 4.09 (d, 1H, J=14 Hz), 3.77 (s, 3H), 3.54 (br, 2H), 3.38 (m, 1H), 3.05 (t, 1H, 12.0 Hz), 2.95 (mbr, 2H), 2.26 (s, 3H), 2.09 (m, 2H), 1.90 (d, 1H, J=8.2 Hz), 1.49 (s, 18H)

MS: m/z=562.5 (M+H).

### EXAMPLE 12

### Preparation of 3-(2-{4-[6-(3,5-di-tert-butylphenyl)pyrazin-2-yl]piperidin 1-yl}-2-oxoethyl)-3H-imidazo[4,5-b]pyridine

### Step 1. tert-butyl 4-(6-chloropyrazin-2-yl)-3,6-dihydropyridine-1(2H)-carboxylate

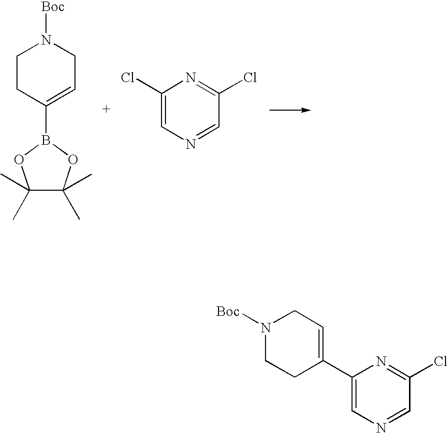

A mixture of boronic ester prepared according to the procedure of Example 2 Step 1 (155 mg, 0.5 mmole), 2,6-dichloropyrazine (12 (111 mg, 0.75 mmole), PdCl₂(PPh₃)₂ (10 mg), acetonitrile (1.5 mL) and Na₂CO₃ solution (2N, 0.5 mL) was degassed and stirred at 100° C. for 5 hrs, and allowed to cool down to room temperature, diluted with 5 mL ethyl acetate, dried over sodium sulfate, filtered and concentrated. Column chromatography yield pale yellowish oil as product.

### Step 2. 3,5-di-tert-butylbenzeneboronic acid

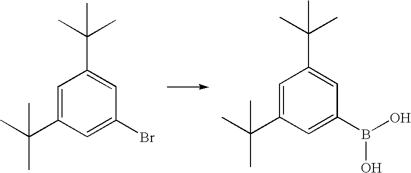

Bromo-3,5-di-tert-butylbenzene (1.3 g, 5 mmole) was dissolved in THF (20 mL), to this solution was slowly added n-BuLi (1.6 M, 3.3 mL) at −78° C., the reaction was stirred at this temperature for 30 min, trimethyl borate (0.75 g, 7.5 mmole) was added all at once, stir over night while the reaction was allowed to slowly warm up to room temperature. Hydrochloric acid (6N, 5 mL) was added and stirred for 3 hrs at room temperature. Water (200 mL) was added, and the precipitate was collected, washed with water (10 mL×3), dried under vacuum to give titled compound.

### Step 3. tert-butyl 4-[6-(3,5-di-tert-butylphenyl)pyrazin-2-yl]-3,6-dihydropyridine-1(2H)-carboxylate

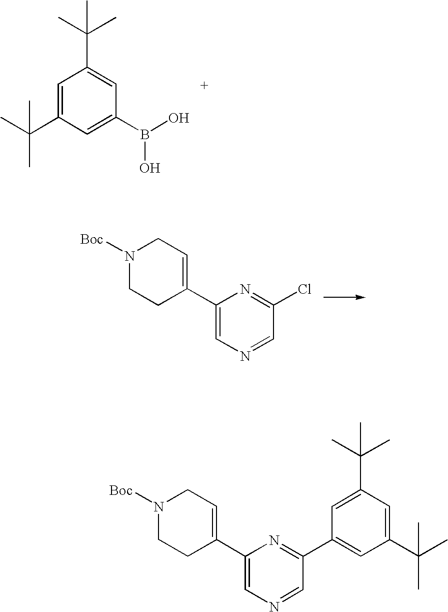

A mixture of 3,5-di-tert-butylbenzeneboronic acid (175 mg, 0.75 mmole), the chloro-pyrazine prepared in Example 12 Step 1 (142 mg, 0.5 mmole), PdCl₂(PPh₃)₂(10 mg), DME (1.5 mL) and Na₂CO₃ solution (2N, 0.5 mL) was degassed and stirred at 100° C. for 5 hrs, and allowed to cool down to room temperature, diluted with 5 mL ethyl acetate, dried over sodium sulfate, filtered and concentrated. Column chromatography yield pale yellowish oil as product.

### Step 4. tert-butyl 4-[6-(3,5-di-tert-butylphenyl)pyrazin-2-yl]piperidine-1-carboxylate

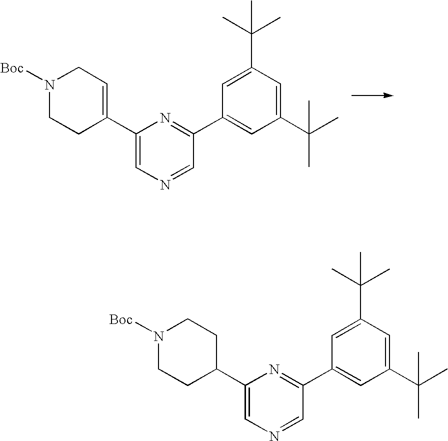

A mixture of alkene prepared according to the procedure of Example 12 Step 3 (12 (50 mg, 0.2 mmole) and Pd/C (4 mg) in Methanol (1.5 mL) was stirred under hydrogen balloon, filtration and concentration generated desired compound.

### Step 5. 3-(2-{4-[6-(3,5-di-tert-butylphenyl)pyrazin-2-yl]piperidin-1-yl}-2-oxoethyl)-3H-imidazo[4,5-b]pyridine

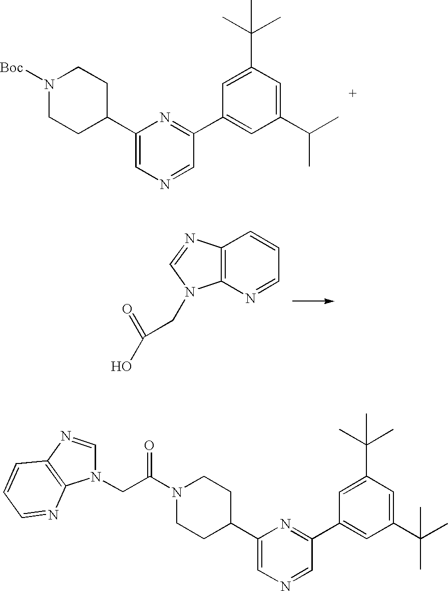

The Boc-protected amine prepared according to the procedure of Example XXX Step 4 (27 mg, 0.06 mmol) was dissolved in trifluoroacetic acid (2 ml) and stirred for 10 minutes. The volatiles were removed i. vac. The residue was dissolved in ethyl acetate, washed with 4:1 water/saturated sodium bicarbonate (40 ml) and extracted (3 times) with ethyl acetate. The combined organic fraction was washed with brine, dried over sodium sulfate, filtered and reduced i. vac.

The unpurified amine (20.7 mg, 0.06) was combined with 1-hydroxybenzotriazole (9.8 mg, 0.07 mmol) and the acid prepared according to the procedure of Example I Step 3 (10.6 mg, 0.06 mmol) and dissolved in dimethylformamide (0.5 mL). Diisopropyl ethyl amine (29.5 mg, 0.23 mmol) and 1-(3-dimethylaminopropyl)-3-ethylcarbodiimide hydrochloride (14.0 mg, 0.07 mmol) were added and the solution allowed to stir overnight. The reaction mixture was diluted with water (6 mL), extracted with diethyl ether, the organic layer was washed with water (2×1 mL), concentrated under vacuo. The residue was redissolved in a mixture of MeOH (0.5 mL) and NaOH (1N aq., 0.07 mL), stir 6 hrs at room temperature, MeoH was removed under vacuo. The mixture was diluted in 2:1 acetonitrile:water (6.2 ml) and purified by RP-18 HPLC (acetonitrile:H₂O 15 minute gradient 10 to 100%:0.1% trifluoroacetic acid) to give the titled compound.

500 MHz NMR (CDCl₃) δ 8.98 (s, 1H), 8.90 (s, 1H), 8.58 (d, 1H, J=4.0 Hz), 8.36 (d, 1H, J=8.0 Hz), 7.85 (d, 2H, J=1.6 Hz), 7.61 (d, 1H, J=1.5 Hz), 7.49 (dd, 1H, J=8.0 Hz, J=2.9 Hz), 5.40 (m, 2H), 4.71 (d, 1H, J=12.5 Hz), 4.19 (d, 1H, J=141 Hz), 3.63 (d, 1H, 13 Hz), 3.21 (m, 1H), 3.02 (t, 1H, 12.0 Hz), 2.26 (d, 1H, J=8 Hz), 2.09 (m, 2H), 1.90 (m, 1H, J=8.2 Hz), 1.43 (s, 18H)

MS: m/z=511.3 (M+H).

### EXAMPLE 13

### Preparation of 1-{2-[4-(3′,5′-di-tert-butylbiphenyl-3-ylpiperidin-1-yl]-2-oxoethyl}-1H-benzimidazole

### Step 1. 3,5-di-tert-butyl-3′-chlorobiphenyl

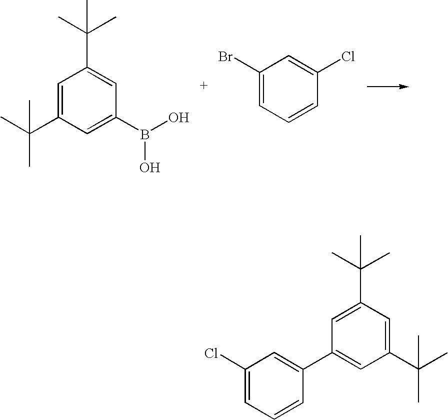

A mixture of 3,5-di-tert-butylbenzeneboronic acid (175 mg, 0.75 mmole), Bromo-3-chlorobenzene (94 mg, 0.5 mmole), PdCl₂(PPh₃)₂ (10 mg), DME (1.5 mL) and Na₂CO₃ solution (2N, 0.5 mL) was degassed and stirred at 100° C. for 5 hrs, and allowed to cool down to room temperature, diluted with 5 mL ethyl acetate, dried over sodium sulfate, filtered and concentrated. Column chromatography yield pale yellowish oil as product

### Step 2. tert-butyl 4-(3′,5′-di-tert-butylbiphenyl-3-yl-3,6-dihydropyridine-1(2H)-carboxylate

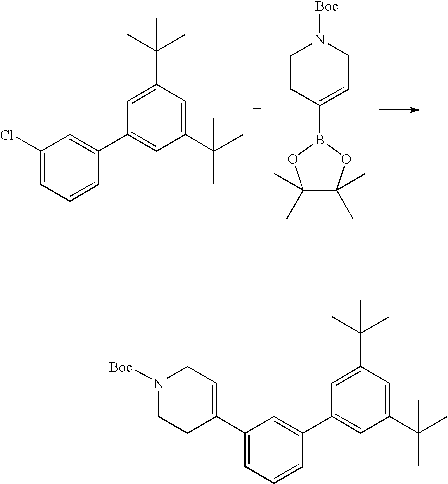

The mixture of the chlorobiphenyl (229 mg, 0.76 mmole), the borate prepared according to the procedure of Example 2 Step 1 (156 mg, 0.5 mmole), PdCl₂(PPh₃)₂(12 mg), DME (1.5 mL) and Na₂CO₃ solution (2N, 0.5 mL) was degassed and stirred at 100° C. for 5 hrs, and allowed to cool down to room temperature, diluted with 5 mL ethyl acetate, dried over sodium sulfate, filtered and concentrated. Column chromatography yield pale yellowish oil as product

### Step 3. tert-butyl 4-(3′,5′-di-tert-butylbiphenyl-3-yl)piperidine-1-carboxylate

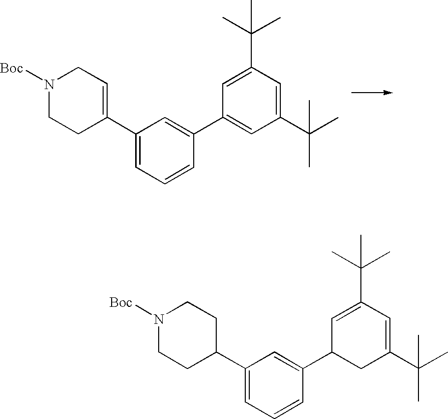

A mixture of the BOC tetrahydropyridine prepared according to the procedure of Example 13 Step 2 (70 mg, 0.28 mmole) and Pd/C (6 mg) in methanol (2 mL) was stirred under hydrogen balloon, filtration and concentration generated desired compound.

### Step 4. 1-{2-[4-(3′,5′-di-tert-butylbiphenyl-3-yl)piperidin-1-yl]-2-oxoethyl}-1H-benzimidazole

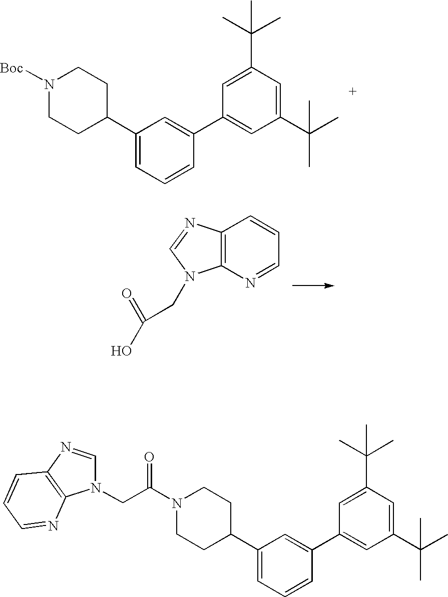

The Boc-protected piperidine prepared according to the procedure of Example 13 Step 3 (28.2 mg, 0.06 mmol) was dissolved in trifluoroacetic acid (2 ml) and stirred for 10 minutes. The volatiles were removed i. vac. The residue was dissolved in ethyl acetate, washed with 4:1 water/saturated sodium bicarbonate (40 ml) and extracted (3 times) with ethyl acetate. The combined organic fraction was washed with brine, dried over sodium sulfate, filtered and reduced i. vac.

The unpurified amine (20.4 mg, 0.06) was combined with 1-hydroxybenzotriazole (9.9 mg, 0.07 mmol) and the acid prepared according to the procedure of Example 1 Step 3 (10.8 mg, 0.06 mmol) and dissolved in dimethylformamide (0.5 mL). Diisopropyl ethyl amine (29.9 mg, 0.23 mmol) and 1-(3-dimethylaminopropyl)-3-ethylcarbodiimide hydrochloride (14.1 mg, 0.07 mmol) were added and the solution allowed to stir overnight. The reaction mixture was diluted with water (6 mL), extracted with diethyl ether, the organic layer was washed with water (2×1 mL), concentrated under vacuo. The residue was redissolved in a mixture of MeOH (0.5 mL) and NaOH (1N aq., 0.07 mL), stir 6 hrs at room temperature, MeoH was removed under vacuo. The mixture was diluted in 2:1 acetonitrile:water (6.2 ml) and purified by RP-18 HPLC (acetonitrile H₂O 15 minute gradient 10 to 100%:0.1% trifluoroacetic acid) to give the titled compound.

500 MHz NMR (CD₃OD) δ 9.07 (s, 1H), 8.58 (d, 1H, J=4.9 Hz), 8.27 (d, 1H, J=8.0 Hz), 7.56 (q, 1H, J=4.8 Hz, J₂=3.8 Hz), 7.38-7.48 (m, br, 6H), 7.27 (d, 1H, J=7.0 Hz), 5.62 (d, 1H, J=18.1 Hz), 5.54 (d, 1H, J=18.1 Hz), 4.60 (d, 1H, J=10 Hz), 3.45 (t, 1H, J=9.1 Hz), 2.95 (t, 1H, J=10.8 Hz), 2.89 (t, 1H, J=9.2 Hz), 2.21 (m, 1H), 1.89 (m, 2H), 1.77 (m, 1H), 1.47 (s, 18H)

MS: m/z=509.2 (M+H).

### EXAMPLE 14

### 3-(2-{4-[6-(3,5-di-tert-butylphenyl)-2-pyridinyl]-1-piperidinyl}-2-oxoethyl)-3H-imidazo[4,5-b]pyridine

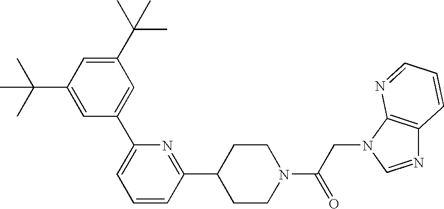

A solution of 2-(3,5-di-tert-butylphenyl)-6-(4-piperidinyl)pyridine (34 mg; 0.097 mmol) in DMF (1 mL) was treated sequentially with 3H-imidazo[4,5-b]pyridin-3-ylacetic acid (34 mg; 0.194 mmol), N-methylmorpholine (27 μL; 0.243 mmol), HOBt (33 mg; 0.243 mmol) and EDC (47 mg; 0.243 mmol). The mixture was stirred at ambient temperature for 2 h. The reaction was partitioned between iPrOAc and aq. NaHCO₃. The organic was dried, filtered and evaporated to a residue. The residue was chromatographed over silica gel (prep. TLC; 20:1 CH₂Cl₂/MeOH). The most polar mobile band was recovered, affording the title compound (30 mg). 500 MHz NMR (CDCl₃): δ 8.40 (d, 1H, J=4.6 Hz), 8.26 (bs, 1H), 8.12 (bd, 1H, J=7.8 Hz), 7.83 (d, 2H, J=1.9 Hz), 7.72 (t, 1H, J=7.8 Hz), 7.59 (d, 1H, J=7.8 Hz), 7.53 (t, 1H, J=1.7 Hz), 7.27 (dd, 1H, J=7.3, 4.8 Hz), 7.10 (d, 1H, J=7.8 Hz), 5.26 (½AB, 1H, J=16.5 Hz), 5.22 (½AB, 1H, J=16.4 Hz), 4.73 (bd, 1H, J=13.5 Hz), 4.18 (bd, 1H, J=13.3 Hz), 3.42 (bt, 1H, J=12.7 Hz), 3.11 (tt, 1H, J=11.5, 3.6 Hz), 2.94 (bt, 1H, J=12.7 Hz), 2.22 (bd, 1H, J=12.5 Hz), 2.13 (bd, 1H, J=13.7 Hz), 1.95 (dhex, 2H, Jₕ=12.3, J_{d}=3.5 Hz), 1.42 (s, 18H). LRMS calc: 509.3 obs: 510.3 (M+H).

### EXAMPLE 15

### 3-[2-(4-{6-[3,5-bis(trifluoromethyl)phenyl]-2-pyridinyl}-1-piperidinyl)-2-oxoethyl]-3H-imidazo[4,5-b]pyridine

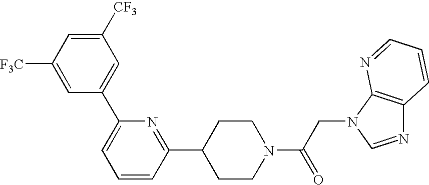

600 MHz NMR (CDCl₃): δ 8.48 (s, 2H), 8.39 (dd, 1H, J=4.7, 1.0 Hz), 8.25 (s, 1H), 8.11 (d, 1H, J=7.9 Hz), 7.93 (s, 1H), 7.80 (t, 1H, J=7.9 Hz), 7.69 (d, 1H, J=7.8 Hz), 7.26 (dd, 1H, J=8.0, 4.7 Hz), 7.22 (d, 1H, J=7.7 Hz), 5.26 (½AB, 1H, J=17.0 Hz), 5.22 (½AB, 1H, J=16.9 Hz), 4.76 (bd, 1H, J=13.4 Hz), 4.20 (bd, 1H, J=13.4 Hz), 3.41 (dt, 1H, Jₜ=13.2, J_{d}=2.5 Hz), 3.11 (tt, 1H, J=12.0, 3.5 Hz), 2.88 (dt, 1H, Jₜ=13.0, 2.6 Hz), 2.19 (bd, 1H, J=13.3 Hz), 2.09 (bd, 1H, J=13.2 Hz), 1.96 (dquart, 1H, J_{q}=12.6, J_{d}=4.0 Hz), 1.87 (dquart, 1H, J_{q}=12.7, J_{d}=3.9 Hz). LRMS calc: 533.2 obs: 534.1 (M+H1).

### EXAMPLE 16

### 2-(3,5-di-tert-butylphenyl)-6-{1-[(3,5-dimethyl-1-pyrazol-1H-yl)acetyl]-4-piperidinyl}pyridine

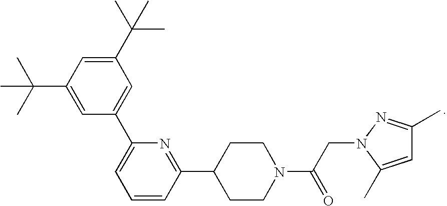

500 MHz NMR (CDCl₃): δ 7.85 (d, 2H, J=1.6 Hz), 7.72 (t, 1H, J=7.8 Hz), 7.60 (d, 1H, J=7.8 Hz), 7.54 (bs, 1H), 7.10 (d, 1H, J=7.8 Hz), 5.90 (bs, 1H), 4.97 (½AB, 1H, J=16.2 Hz), 4.92 (½AB, 1H, J=16.2 Hz), 4.74 (bd, 1H, J=13.3 Hz), 4.14 (bd, 1H, J=13.2 Hz), 3.30 (bt, 1H, J=11.9 Hz), 3.07 (tt, 1H, J=11.7, 3.4 Hz), 2.88 (dt, 1H, Jₜ=13.0, J_{d}=2.3 Hz), 2.29 (s, 3H), 2.26 (s, 3H), 2.13 (bt, 1H, J=14.3 Hz), 1.92-1.80 (m, 2H), 1.44 (s, 18H). LRMS calc: 486.3 obs: 487.4 (M+H).

### EXAMPLE 17

### 2-(3,5-di-tert-butylphenyl)-6-{1-[(3,5-dimethyl-1-1,2,4-triazol-1H-yl)acetyl]-4-piperidinyl}pyridine

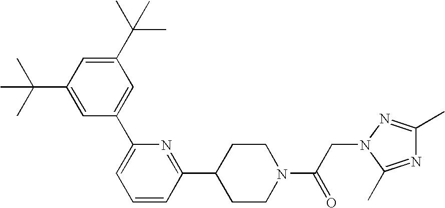

500 MHz NMR (CDCl₃): δ 7.82 (d, 2H, J=1.8 Hz), 7.71 (t, 1H, J=7.8 Hz), 7.59 (d, 1H, J=7.7 Hz), 7.52 (t, 1H, J=1.7 Hz), 7.09 (d, 1H, J=7.6 Hz), 4.94 (bs, 2H), 4.70 (bd, 1H, J=13.5 Hz), 4.05 (bd, 1H, J=13.5 Hz), 3.34 (dt, 1H, Jₜ=14.2, J_{d}=2.1 Hz), 3.08 (tt, 1H, J=11.4, 3.5 Hz), 2.90 (dt, 1H, Jₜ=13.1, J_{d}=1.8 Hz), 2.44 (s, 3H), 2.36 (s, 3H), 2.19 (bd, 1H, J=13.1 Hz), 2.12 (bd, 1H, J=13.1 Hz), 1.94-1.85 (m, 2H), 1.41 (s, 18H). LRMS calc: 487.3 obs: 488.3 (M+H).

### EXAMPLE 18

### 3-(2-{4-[6-(3,5-di-tert-butyl-4-methoxyphenyl)-2-pyridinyl]-1-piperidinyl}-2-oxoethyl)-3H-imidazo[4,5-b]pyridine

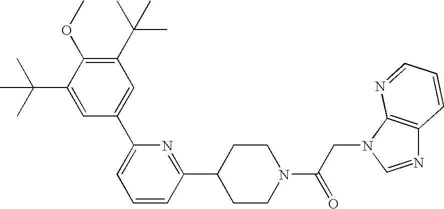

500 MHz NMR (CDCl₃): δ 8.40 (d, 1H, J=3.9 Hz), 8.26 (bs, 1H), 8.13 (bd, 1H, J=7.7 Hz), 7.90 (s, 2H), 7.70 (t, 1H, J=7.8 Hz), 7.54 (d, 1H, J=7.7 Hz), 7.27 (dd, 1H, J=7.8, 4.8 Hz), 7.07 (d, 1H, J=7.7 Hz), 5.26 (½AB, 1H, J=16.2 Hz), 5.22 (½AB, 1H, J=16.4 Hz), 4.71 (bd, 1H, J=13.1 Hz), 4.19 (bd, 1H, J=13.5 Hz), 3.75 (s, 3H), 3.43 (dt, 1H, Jₜ=12.4, J_{d}=2.5 Hz), 3.09 (tt, 1H, J=11.5, 3.7 Hz), 2.95 (dt, 1H, Jₜ=13.1, J_{d}=2.0 Hz), 2.22 (bd, 1H, J=13.1 Hz), 2.13 (bd, 1H, J=13.0 Hz), 1.96 (dquart, 1H, J_{q}=12.8, J_{d}=3.6 Hz), 1.90 (dquart, 1H, J_{q}=13.2, J_{d}=4.0 Hz), 1.52 (s, 18H). LRMS calc: 539.3 obs: 540.4 (M+H).

### EXAMPLE 19

### 2-(3,5-di-tert-butyl-4-methoxyphenyl)-6-{1-[(3,5-dimethyl-1H-pyrazol-1-yl)acetyl]-4-piperidinyl}pyridine

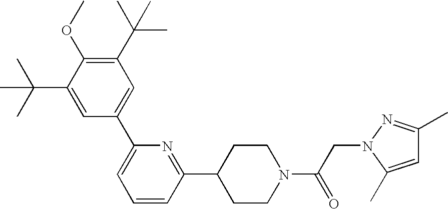

500 MHz NMR (CDCl₃): δ 7.90 (s, 2H), 7.69 (t, 1H, J=7.8 Hz), 7.54 (d, 1H, J=7.7 Hz), 7.05 (d, 1H, J=7.5 Hz), 5.88 (bs, 1H), 4.95 (½AB, 1H, J=16.0 Hz), 4.91 (½AB, 1H, J=16.1 Hz), 4.71 (bd, 1H, J=13.1 Hz), 4.12 (bd, 1H, J=13.3 Hz), 3.75 (s, 3H), 3.28 (dt, 11, Jₜ=12.3, J_{d}=2.3 Hz), 3.04 (tt, 1H, J=11.7, 3.6 Hz), 2.86 (dt, 1H, Jₜ=12.8, J_{d=2.5} Hz), 2.27 (s, 3H), 2.24 (s, 3H), 2.11 (bt, 2H, J=15.6 Hz), 1.92-1.80 (m, 2H), 1.52 (s, 18H). LRMS calc: 516.4 obs: 517.4 (M+H).

### EXAMPLE 20

### 2-(3,5-di-tert-butyl-4-methoxyphenyl)-6-{1-[(2,4-dimethyl-1H-imidazol-1-yl)acetyl]-4-piperidinyl}pyridine

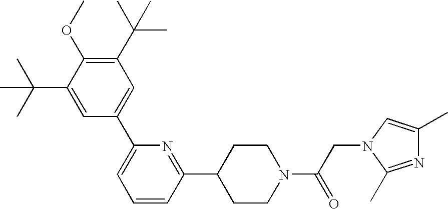

500 MHz NMR (CDCl₃): δ 7.90 (s, 2H), 7.69 (t, 1H, J=7.8 Hz), 7.54 (d, 1H, J=7.8 Hz), 7.06 (d, 1H, J=7.7 Hz), 6.57 (bs, 1H), 4.72-4.66 (bd overlapping AB, 3H total), 3.94 (bd, 1H, J=13.5 Hz), 3.75 (s, 3H), 3.33 (bt, 1H, J=11.7 Hz), 3.07 (tt, 1H, J=11.4, 3.7 Hz), 2.92 (bt, 1H, J=12.6 Hz), 2.38-2.34 (s overlapping bd, 4H total), 2.22 (s, 3H), 2.11 (bd, 1H, J=11.2 Hz), 1.90 (vbquart, 2H, J=12.3 Hz), 1.51 (s, 18H). LRMS calc: 516.4 obs: 517.4 (M+H).

### EXAMPLE 21

### 3-[2-(4-{4-[3,5-bis(trifluoromethyl)phenyl]-2-pyrimidinyl}-1-piperidinyl)-2-oxoethyl]-3H-imidazo[4,5-b]pyridine

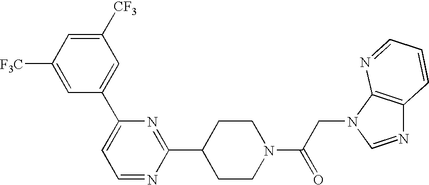

500 MHz NMR (CDCl₃): δ 8.87 (d, 1H, J=5.2 Hz), 8.57 (s, 2H), 8.41 (dd, 1H, J=4.8, 1.4 Hz), 8.26 (s, 1H), 8.12 (dd, 1H, J=8.0, 1.4 Hz), 8.05 (s, 1H), 7.68 (d, 1H, J=5.2 Hz), 7.27 (dd, 1H, J=8.0, 4.8 Hz), 5.27 (½AB, 1H, J=16.2 Hz), 5.23 (½AB, 1H, J=16.4 Hz), 4.72 (bd, 1H, J=13.5 Hz), 4.20 (bd, 1H, J=13.5 Hz), 3.45 (dt, 1H, Jₜ=13.5, J_{d}=2.7 Hz), 3.32 (tt, 1H, J=11.4, 3.6 Hz), 2.97 (dt, 1H, Jₜ=13.0, 2.9 Hz), 2.29 (bd, 1H, J=12.6 Hz), 2.23 (bd, 1H, J=12.2 Hz), 2.09 (dquart, 1H, J_{q}=12.8, J_{d}=3.9 Hz), 1.97 (dquart, 1H, J_{q}=13.0, J_{d}=4.1 Hz). LRMS calc: 534.2 obs: 535.1 (M+H).

### EXAMPLE 22

### 3-(2-{4-[6-(3,5-di-tert-butylphenyl)-2-pyridinyl]-1-piperazinyl}-2-oxoethyl)-3H-imidazo[4,5-b]pyridine

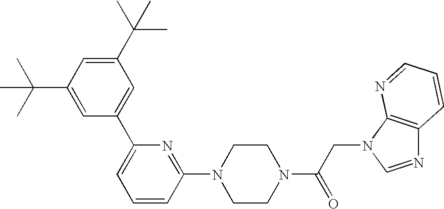

500 MHz NMR (CDCl₃): δ 8.39 (dd, 1H, J=4.8, 1.4 Hz), 8.37 (s, 1H), 8.12 (dd, 1H, J=8.0, 1.3 Hz), 7.87 (d, 1H, J=1.8 Hz), 7.66 (t, 1H, J=8 Hz), 7.5 (t, 1H, J=3.4 Hz), 7.37 (dd, 1H, J=8, 4.8 Hz), 7.21 (d, 1H, J=7.5 Hz), 6.80 (d, 1H, J=8.4 Hz), 5.43 (s, 2H), 3.89 (s, 4H), 3.77 (q, 2H, J=3.4 Hz), 3.7 (q, 2H, J=3.3 Hz), 1.39 (s, 18H).

### Assays for Determining Biological Activity

### Human CXCR3 Receptor.

The compounds claimed here are assayed for affinity and functional potency at the CXCR3 receptor using the assays described below.

Since the expression of CXCR3 on naive T cells is low, PBMC's were cultured in the presence of a mixture of superantigens to provide primary cells with sufficient CXCR3 expression to use routinely in binding and functional assays. Briefly, mononuclear cells were enriched from buffy coats obtained from a local blood bank by centrifugation over Ficoll-Hypaque. Residual red blood cells were lysed in hypotonic buffer, (ACK), cells were washed with PBS and resuspended in media (RPMI containing 10% FBS, 2 mM glutamine, MEM non essential amino acids and sodium pyruvate) containing 500 Units/ml of IL-2 and 0.5 ng/ml SE cocktail (containing equal amounts of SEA, SEB, SEC1, SED and SEE all from Toxin Technology). After several days in culture, cells were switched to fresh media containing 500 units/ml of IL-2 and cultures were maintained at 2-4 million cells/ml for up to 21 days.

### Binding Assay.

Inhibition of binding of CXCL10 or CXCL11 to human CXCR3 was measured in whole cells, using superantigen activated T cells (SE-T) at day 7-14 post stimulation. Binding of ¹²⁵I-IP-10 (2200 Ci/mmol, typically 20 pM) in the presence of unlabeled ligands was initiated by adding intact T cells (200,000 cells/assay) in a total assay volume of 250 μl (containing 50 mM HEPES, pH 7.2, 5 mM MgCl2, 1 mM CaCl2 and 0.5% BSA. Binding of ¹²⁵I-I-TAC (2200 Ci/mmol, 20 μM) was performed as described for IP-10 except for the addition of 0.15M NaCl to the binding buffer. After incubation at room temperature for 2 hours with shaking, the reaction was terminated by filtering through a 0.1% polyethylenimine (Sigma) soaked GF/C filter plate (Packard) using a Packard Filtermate cell harvester and the plate washed with approximately 750 μl of 50 mM HEPES (Sigma), pH 7.2, 500 mM NaCl chilled to 4° C. The plates were dried; scintillant added and counted on a Packard TopCount. Non-specific binding was measured in the presence of 1 μM ligand (IP-10 or I-TAC). Binding results were analyzed using Microsoft Excel and GraphPad Prism software.

The Examples disclosed herein were tested in the above receptor binding assay and demonstrated an IC₅₀ ranging from 4 to 4000 nM against ¹²⁵I-IP-10 and an IC₅₀ ranging from 50 to 1800 nM against ¹²⁵I-I-TAC.

### Functional Assays.

The functional potency of the claimed compounds was assessed by measuring inhibition of the chemotaxis of leukocytes in response to CXCR3 ligands. A modified Boyden chamber chemotaxis system (ChemoTx™, NeuroProbe, Gaithersburg, Md.), consisting of a 96-well microplate and a filter (6.0-mm diameter, 5-μ pore size), coated on the bottom with fibronectin (50 μl of a 10 μg/ml solution, then air-dried), was used for chemotaxis measurements. Briefly, aliquots of human T cells (day 14 to day 17 post activation) were washed and resuspended at 1×107 cells/ml in warm (37° C.) Banks' balanced saline solution (HBSS)/bovine serum albumin [(BSA); HBSS without phenol red, calcium, or magnesium (Mediatec)+0.01% BSA] and loaded with Calcein-AM (Molecular Probes) at a concentration of 2 μM for 30 min at 37° C. The cells were washed again in HBSS/BSA and resuspended in RPMI/BSA [RPMI without phenol red (Mediatec)+0.5% BSA+1% dimethyl sulfoxide] to a concentration of 6×106 cells/ml. To initiate the chemotaxis, chemokines were diluted in warm (37° C.) RPMI/BSA and added in 30 μl to the bottom of the microplate before affixing the filter to the unit. Aliquots (50 μl) of the Calcein-loaded T cells were then added to the top of the filter over each individual well. The microplates were subsequently incubated for 1 h at 37° C. Remaining cells were suctioned off the top of the filter. The filter was rinsed with PBS and wiped with a rubber squeegee. The plate with filter intact was read in a Cytofluor™ II fluorometer (PerSeptive Biosystems, Foster City, Calif.). For assay of antagonists, compounds were diluted in DMSO and added to both cells and ligand in a final DMSO concentration of 0.5%.

The Examples disclosed herein were tested in the above assay against both IP-10 and I-TAC. The Examples demonstrated an IC₅₀ ranging from 0.5 to 600 nM against IP-10 and typically a somewhat higher IC₅₀ ranging from 25 to 1700 nM against I-TAC.

What is claimed is:

1. A compound of Formula I 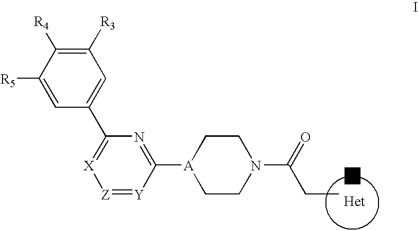
or a pharmaceutically acceptable salt thereof, wherein:
A is CH or N;
one of X, Y and Z is N or CH, the other of X, Y and Z are CH;
R₃ is selected from the group consisting of: C₁₋₄alkyl, —CF₃, —OCF₃ and —S(O)ₙCF₃, wherein n is 0 or 2;
R₄ is selected from the group consisting of: H, halo, —OH, —OCH₃, —OCH₂CF₃ and —CF₃;
or R₃ and R₄ may be joined together with the carbon atoms to which they are attached to form a five- or six-membered monocyclic ring, said rings tetra-substituted with methyl groups as follows: 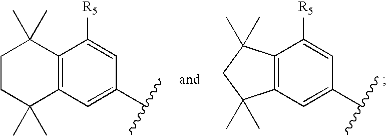
R₅ is selected from the group consisting of: C₁₋₄alkyl, C₃₋₆cycloalkyl, CF₃, —CF₂CH₃, —OCF₃ and —SCF₃; and 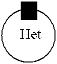
is a 5 membered non-aromatic or aromatic ring or a 9 membered fused bicyclic partially aromatic or aromatic ring, each ring containing at least 1 nitrogen atom and optionally up to 3 additional heteroatoms selected from S, O and N, said rings optionally substituted with 1 to 3 substituents independently selected from the group consisting of: oxo, hydroxy, carboxy, —CF₃, halo, —S(O)ₚ—CH₃, phenyl, C₁₋₃alkoxy and C₁₋₃alkyl, said C₁₋₃alkyl optionally substituted with carboxy or hydroxy; and
p is 0, 1 or 2.

2. The compound according to claim 1 wherein: 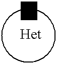
is selected from the group consisting of: 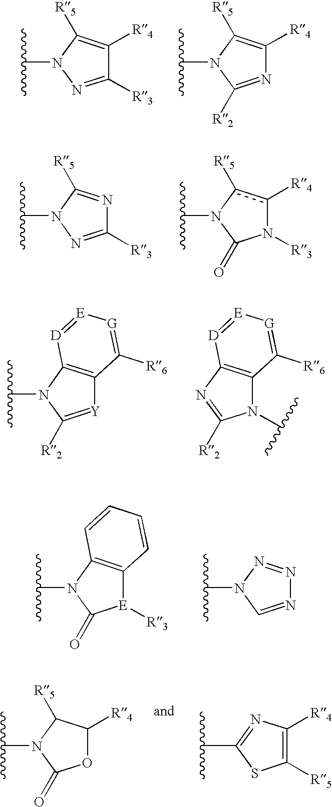
wherein D, E and G are independently C or N,
R″₂, R″₃, R″₄ and R″₅ are independently selected from the group consisting of: —H, carboxy, —CF₃, halo, methylthio, methylsulfonyl, phenyl, C₁₋₃alkoxy and C₁₋₃alkyl, said C₁₋₃alkyl optionally substituted with carboxy or hydroxy,
R″₆ is H or OH, and
------ is an optional double bond.

3. The compound according to claim 1 wherein A is N.

4. The compound according to claim 1 wherein A is CH.

5. The compound according to claim 4 wherein X, Y and Z are CH.

6. The compound according to claim 4 wherein X is N and Y and Z are CH.

7. The compound according to claim 4 wherein Y is N and X and Z are CH.

8. The compound according to claim 4 wherein Z is N and X and Y are CH.

9. The compound according to claim 1 wherein R₃ and R₅ are tert-butyl.

10. The compound according to claim 1 wherein R₃ and R₅ are CF₃.

11. The compound according to claim 1 wherein: 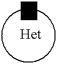
is selected from the group consisting of:

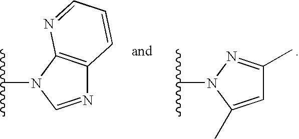

12. The compound according to claim 1 wherein:
A, X, Y and Z are CH;
R₃ and R₅ are tert-butyl or R₃ and R₅ are CF₃; and
R₄ is selected from H and —OCH₃.

13. The compound according to claim 10 wherein: 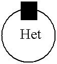
is selected from the group consisting of:

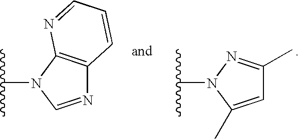

14. A compound according to claim 1 selected from the following group:
1) 3-(2-(4-[6-(3,5-di-tert-butylphenyl)pyridine-2-yl]piperazin-1-yl)-2-oxoethyl)-3-H-imdazo[4,5-b]pyridine;
2) 3-(2-{4-[6-(3,5-di-tert-butylphenyl)-2-pyridinyl]-1-piperidinyl}-2-oxoethyl)-3H-imidazo[4,5-b]pyridine;
3) 2-(3,5-di-tert-butylphenyl)-6-{1-[(3,5-dimethyl-1-pyrazol-1H-yl)acetyl]-4-piperidinyl}pyridine;
4) 2-(3,5-di-tert-butylphenyl)-6-{1-[(3,5-dimethyl-1-1,2,4-triazol-1H-yl)acetyl]-4-piperidinyl}pyridine;
5) 3-[2-(4-{6-[3,5-bis(trifluoromethyl)phenyl]-2-pyridinyl}-1-piperidinyl)-2-oxoethyl]-3H-imidazo[4,5-b]pyridine;
6) 3-(2-{4-[6-(3,5-di-tert-butyl-4-methoxyphenyl)-2-pyridinyl]-1-piperidinyl}-2-oxoethyl)-3H-imidazo[4,5-b]pyridine;
7) 2-(3,5-di-tert-butyl-4-methoxyphenyl)-6-{1-[(3,5-dimethyl-1H-pyrazol-1-yl)acetyl]-4-piperidinyl}pyridine;
8) 2-(3,5-di-tert-butyl-4-methoxyphenyl)-6-{1-[(2,4-dimethyl-1H-imidazol-1-yl)acetyl]-4-piperidinyl}pyridine;
9) 3-[2-(4-{4-[3,5-bis(trifluoromethyl)phenyl]-2-pyrimidinyl}-1-piperidinyl)-2-oxoethyl]-3H-imidazo[4,5-b]pyridine;
10) 3-(2-{4-[2-(3,5-di-tert-butyl-4-methoxyphenyl)pyrimidin-4-yl]piperidin-1-yl}-2-oxoethyl)-3H-imidazo[4,5-b]pyridine,
11) [1-(2-{4-[2-(3,5-di-tert-butyl-4-methoxyphenyl)pyrimidin-4-yl]piperidin-1-yl}-2-oxoethyl)-5-methyl-1H-pyrazol-3-yl]acetic acid;
12) 3-(2-{4-[6-(3,5-di-tert-butylphenyl)pyrazin-2-yl]piperidin-1-yl}-2-oxoethyl)-3H-imidazo[4,5-b]pyridine;
13) 1-{2-[4-(3′,5-di-tert-butylbiphenyl-3-yl)piperidin-1-yl]-2-oxoethyl}-1H-benzimidazole;
14) 3-(2-{4-[6-(3,5-di-tert-butylphenyl)-2-pyridinyl]-1-piperidinyl}-2-oxoethyl)-3H-imidazo[4,5-b]pyridine
15) 3-[2-(4-{6-[3,5-bis(trifluoromethyl)phenyl]-2-pyridinyl}-1-piperidinyl)-2-oxoethyl]-3H-imidazo[4,5-b]pyridine;
16) 2-(3,5-di-tert-butylphenyl)-6-{1-[(3,5-dimethyl-1-pyrazol-1H-yl)acetyl]-4-piperidinyl}pyridine;
17) 2-(3,5-di-tert-butylphenyl)-6-{1-[(3,5-dimethyl-1-1,2,4-triazol-1H-yl)acetyl]-4-piperidinyl}pyridine;
18) 3-(2-{4-[6-(3,5-di-tert-butyl-4-methoxyphenyl)-2-pyridinyl]-1-piperidinyl}-2-oxoethyl)-3H-imidazo[4,5-b]pyridine;
19) 2-(3,5-di-tert-butyl-4-methoxyphenyl)-6-{1-[(3,5-dimethyl-1H-pyrazol-1-yl)acetyl]-4-piperidinyl}pyridine;
20) 2-(3,5-di-tert-butyl-4-methoxyphenyl)-6-{1-[(2,4-dimethyl-1H-imidazol-1-yl)acetyl]-4-piperidinyl}pyridine;
21) 3-[2-(4-{4-[3,5-bis(trifluoromethyl)phenyl]-2-pyrimidinyl}-1-piperidinyl)-2-oxoethyl]-3H-imidazo[4,5-b]pyridine; and
22) 3-(2-{4-[6-(3,5-di-tert-butylphenyl)-2-pyridinyl]-1-piperazinyl}-2-oxoethyl)-3H-imidazo[4,5-b]pyridine,
or a pharmaceutically acceptable salt of any of the aforementioned.

15. A pharmaceutical composition comprising a compound according to claim 1 in combination with a pharmaceutically acceptable carrier.

16. A method for treating a disease or condition mediated by the CXCR3 chemokine receptor comprising administering to a patient in need of such treatment a therapeutically effective amount of a compound according to claim 1.

17. The method according to claim 14 wherein the disease or condition is selected from the group consisting of: acute and chronic transplant rejection, psoriasis, rheumatoid arthritis and multiple sclerosis.
